(19) United States
(12) Patent Application Publication
(10) Pub. No.: US 2026/0158048 A1
(43) Pub. Date: Jun. 11, 2026

(54) METHODS OF TREATMENT

(71) Applicant: Rigel Pharmaceuticals, Inc., South San Francisco, CA (US)

(72) Inventors: Andrew Chow, Walnut Creek, CA (US); Toufigh Gordi, San Carlos, CA (US); Lisa Rojkjaer, Rancho Santa Fe, CA (US); Lucy Yan, San Mateo, CA (US)

(21) Appl. No.: 19/198,985

(22) Filed: May 5, 2025

Related U.S. Application Data
(60) Provisional application No. 63/729,256, filed on Dec. 6, 2024.

(51) Int. Cl. A61K 31/675; A61K 9/00; A61K 9/20; A61K 9/48; A61P 35/00

(52) U.S. Cl. CPC A61K 31/675; A61K 9/0053; A61K 9/20; A61K 9/48; A61P 35/00

(57) ABSTRACT

Disclosed herein are methods for treating, managing, or ameliorating a disease selected from myelodysplastic syndrome (MDS), myeloproliferative neoplasms (MPN), MPN/MDS, clonal cytopenia of undetermined significance (CCUS), chronic myeloid leukemia (CML) and acute myeloid leukemia (AML). The methods comprise administering to a subject in need thereof a therapeutically effective amount of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate or a stereoisomer or mixture of stereoisomers, a pharmaceutically acceptable salt, tautomer, solvate, hydrate, co-crystal, clathrate, or polymorph thereof.

### RELATED APPLICATIONS

This application claims the benefit of U.S. Provisional Application No. 63/729,256, filed Dec. 6, 2024, the disclosure of which is incorporated herein by reference in its entirety.

### FIELD

Provided herein are methods for treating, managing, or ameliorating a disease selected from myelodysplastic syndrome (MDS), myeloproliferative neoplasms (MPN), MPN/MDS, clonal cytopenia of undetermined significance (CCUS), chronic myeloid leukemia (CML) and acute myeloid leukemia (AML). The methods comprise administering a therapeutically effective amount of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate or a stereoisomer or mixture of stereoisomers, a pharmaceutically acceptable salt, tautomer, solvate, hydrate, co-crystal, clathrate, or polymorph thereof.

### BACKGROUND

Interleukin-1 receptor-associated kinases (IRAKs) are important mediators of signaling processes, such as toll-like receptors (TLR) and interleukin-1 receptor (IL-1R) signaling processes. IRAKs have been implicated in modulating signaling networks that control inflammation, apoptosis, and cellular differentiation. Four IRAK genes have been identified in the human genome (IRAK1, IRAK2, IRAK3 and IRAK4), and studies have revealed distinct, non-redundant biological roles. IRAK1 and IRAK4 have been shown to exhibit kinase activity. Compound 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate or a stereoisomer or mixture of stereoisomers, a pharmaceutically acceptable salt, tautomer, solvate, hydrate, co-crystal, clathrate, or polymorph thereof (Compound 1) has been reported as an IRAK inhibitor and/or useful for delivering an IRAK inhibitor having a therapeutic use in treatment of diseases where IRAKs are implicated. There is a continuing need to develop therapeutically efficacious dosages for and methods for treatment using Compound 1.

### SUMMARY

Disclosed herein are methods for treating, preventing, managing, and/or ameliorating a disease by administering Compound 1 to a subject, wherein the disease is myelodysplastic syndrome (MDS), myeloproliferative neoplasms (MPN), MPN/MDS, clonal cytopenia of undetermined significance (CCUS), chronic myeloid leukemia (CML) or acute myeloid leukemia (AML).

In one embodiment, provided herein are methods for treating, managing, or ameliorating a disease comprising administering to a subject having the disease Compound 1, wherein Compound 1 is orally administered to the subject in an amount of about 100 mg to about 2000 mg per day, and the disease is myelodysplastic syndrome (MDS), myeloproliferative neoplasms (MPN), MPN/MDS, clonal cytopenia of undetermined significance (CCUS), chronic myeloid leukemia (CML) or acute myeloid leukemia (AML). In one embodiment, provided herein are methods for treating, managing, or ameliorating a disease comprising administering to a subject having the disease Compound 1, wherein Compound 1 is orally administered to the subject in an amount of about 100 mg to about 1000 mg per day, and the disease is myelodysplastic syndrome (MDS), myeloproliferative neoplasms (MPN), MPN/MDS, clonal cytopenia of undetermined significance (CCUS), chronic myeloid leukemia (CML) or acute myeloid leukemia (AML).

In one embodiment, the methods comprise administering to a subject having a myelodysplastic syndrome a therapeutically effective amount of compound 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate, which has the following structure:

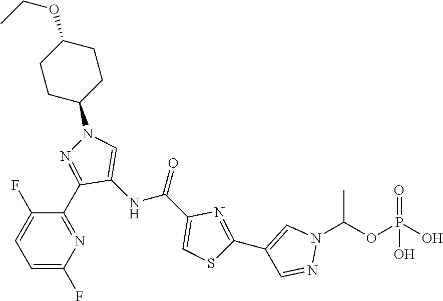 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]

or a stereoisomer or mixture of stereoisomers, a pharmaceutically acceptable salt, tautomer, solvate, hydrate, co-crystal, clathrate, or polymorph thereof (Compound 1). In one embodiment, the methods comprise orally administering Compound 1 to the subject in an amount of about 100 mg to about 2000 mg per day.

In one embodiment, Compound 1 is an inorganic base salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate disodium salt (Compound IA). In one embodiment, Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt (Compound 1B). In one embodiment, Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate (Compound 1C).

In one embodiment, the myelodysplastic syndrome is a higher-risk myelodysplastic or a lower-risk myelodysplastic syndrome.

In one embodiment, the methods provided herein comprise treating a subject who is relapsed, refractory/resistant, intolerant, or has an inadequate response to one or more prior therapies of a myelodysplastic syndrome. In one embodiment, the prior therapy comprises a treatment with an erythropoietin, luspatercept, or a hypomethylating agent.

In one embodiment, the methods comprise administering Compound 1 in an amount corresponding to about 150 mg, about 200 mg, about 250 mg, about 300 mg, about 350 mg, about 375 mg, about 400 mg, about 450 mg, about 500 mg, about 550 mg, about 600 mg, about 650 mg, about 700 mg, about 750 mg, about 800 mg, about 850 mg, about 900, about 950 mg or about 1000 mg daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

In one embodiment, the methods comprise administering Compound 1 in an amount corresponding to about 150 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 250 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 300 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 350 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 375 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 400 mg once daily of 1-(4-(4-((3-(3,6-difluoropyridin-2-yl)-1-((1 r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 500 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 750 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 1000 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

In one embodiment, the methods comprise administering Compound 1 in an amount corresponding to about 150 mg twice daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 200 mg twice daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 250 mg twice daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 375 mg twice daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 400 mg twice daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 500 mg twice daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

In one embodiment, Compound 1 is administered to the subject with food. In one embodiment, Compound 1 is administered to the subject without food.

In one embodiment, the methods provided herein further comprise administering a therapeutically effective amount of a second active agent or a supportive care therapy.

These and other aspects of the subject matter described herein will become evident upon reference to the following detailed description.

### BRIEF DESCRIPTION OF DRAWINGS

FIG. 1 provide plots of observed N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide (Compound 2) concentrations during the PK sampling window at steady state and presumed concentrations resulting in various degrees of cytokine inhibition, dose levels 1-4.

FIG. 2 provides plots of predicted Compound 2 concentrations at steady state in the planned Compound 2 dosing cohorts.

FIG. 3 provides a plot showing dose-normalized Cₘₐₓ and AUC_{0-inf} estimates of N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide (Compound 2) and 3-(3,6-difluoropyridin-2-yl)-1-((1r,4r)-4-ethoxycyclohexyl)-1H-pyrazol-4-amine (Compound 3) after a single dose of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

FIGS. 4A and 4B provides trough concentrations for N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide and Compound 3 after 500 mg and 960 mg BID doses of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

FIG. 5 provides schematic presentation of the nonlinear mixed-effect pharmacokinetic model of N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide and Compound 3 after 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate administrations.

FIG. 6 provides visual predictive check plots stratified by analyte for all subjects in the integrated model, for N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide and Compound 3 after 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate administrations.

FIG. 7 provides plots of simulated vs. observed concentrations of N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide and Compound 3 after a single 750 mg dose of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

FIG. 8 provides a chart showing red blood cell transfusion frequency by dose level of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

### DETAILED DESCRIPTION

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, percentages, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

The term Compound 1 refers to “1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate, which has the following structure:

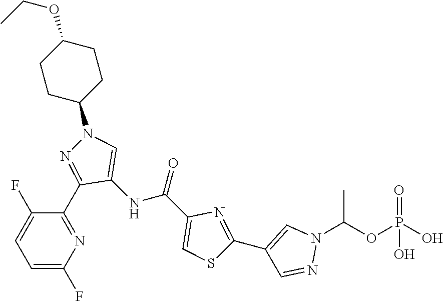 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]

or a stereoisomer or mixture of stereoisomers, a pharmaceutically acceptable salt, tautomer, solvate, hydrate, co-crystal, clathrate, or polymorph thereof”.

The term “Compound 2” refers to N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide, which is the active moiety of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

The term “Compound 3” refers to 3-(3,6-difluoropyridin-2-yl)-1-((1r,4r)-4-ethoxycyclohexyl)-1H-pyrazol-4-amine, which is a metabolite of N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide.

The term “subject” or “patient” refers to an animal, including, but not limited to, a mammal, including a primate (e.g., human), cow, sheep, goat, horse, dog, cat, rabbit, rat, or mouse. The terms “subject” and “patient” are used interchangeably herein in reference, for example, to a mammalian subject, such as a human subject.

In one embodiment, the subject has myelodysplastic syndrome (MDS), including, but is not limited to the following subtypes of MDS. The terms “myelodysplastic syndromes” or “myelodysplastic syndrome” refer to hematological conditions characterized by abnormalities in the production of one or more of the cellular components of blood (red cells, white cells (other than lymphocytes) and platelets (or their progenitor cells, megakaryocytes)), and includes the following disorders: refractory anemia (RA); RA with ringed sideroblasts (RARS); RA with excess of blasts (RAEB); refractory cytopenia with multilineage dysplasia (RCMD), refractory cytopenia with unilineage dysplasia (RCUD); unclassifiable myelodysplastic syndrome (MDS-U), myelodysplastic syndrome associated with an isolated del(5q) chromosome abnormality, therapy-related myeloid neoplasms and chronic myelomonocytic leukemia (CMML). The MDS as used herein also includes very low risk, low risk, intermediate risk, high risk and very high risk MDS. In some embodiments, the MDS is primary or de novo MDS. In other embodiments, the MDS is secondary.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a patient with such a disease or disorder. In some embodiments, the terms refer to the administration of a compound provided herein, with or without other additional active agent, after the onset of symptoms of the particular disease. In one embodiment, the disease is myelodysplastic syndrome (MDS), myeloproliferative neoplasms (MPN), MPN/MDS, clonal cytopenia of undetermined significance (CCUS), chronic myeloid leukemia (CML) or acute myeloid leukemia (AML). In one embodiment, the disease can be relapsed, refractory or resistant to conventional therapy. In one embodiment, the disease is MDS, including, a subtype of MDS discussed elsewhere herein.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of diseases or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.” In one embodiment, the disease is leukemia, including, but is not limited to, chronic lymphocytic leukemia, chronic myelocytic leukemia, acute lymphoblastic leukemia, acute myeloid leukemia, and acute myeloblastic leukemia. In one embodiment, the leukemia can be relapsed, refractory or resistant to conventional therapy. In one embodiment, the disease is MDS, including, a subtype of MDS discussed above.

As used herein, and unless otherwise specified, the terms “manage,” “managing” and “management” refer to preventing or slowing the progression, spread or worsening of a disease or disorder, or of one or more symptoms thereof. Often, the beneficial effects that a patient derives from a prophylactic and/or therapeutic agent do not result in a cure of the disease or disorder. In this regard, the term “managing” encompasses treating a patient who had suffered from the particular disease in an attempt to prevent or minimize the recurrence of the disease, or lengthening the time during which the remains in remission. In one embodiment, the disease is leukemia, including, but not limited to, chronic lymphocytic leukemia, chronic myelocytic leukemia, acute lymphoblastic leukemia, acute myeloid leukemia, and acute myeloblastic leukemia. In one embodiment, the leukemia can be relapsed, refractory or resistant to conventional therapy. In one embodiment, the disease is MDS, including, a subtype of MDS discussed above.

The term “adverse effect” is used according to its ordinary and common meaning in the art and as used herein can refer to a specific condition associated with treatment, prevention, management, or amelioration of a disease described herein resulting from treatment with a compound or composition described herein. One such adverse effect is the onset of neutropenia. Neutropenia can result from damage to bone marrow, and refers to any condition causing inhibition, elimination, or disruption (directly or indirectly) of neutrophil production and/or maturation.

The term “refractory or resistant” refers to a circumstance where a subject or a mammal, even after intensive treatment, has residual cancer cells in his body. In one embodiment, “refractory” means the disease or condition does not respond to treatment. The disease or condition may be resistant at the beginning of treatment or it may become resistant during treatment. The term “relapsed” refers to the return of a disease or the signs and symptoms of a disease after a period of improvement. In one embodiment, “relapsed” refers to the return of a disease or the signs and symptoms of a disease after primary treatment of the disease ends.

The term “QD” refers to a once daily dose administration.

The term “BID” refers to a twice daily dose administration.

The terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

As used herein, “pharmaceutically acceptable excipient” refers to a substance, other than the active ingredient or prodrug thereof, that is included in a formulation of the active ingredient. As used herein, an excipient may be incorporated within particles of a pharmaceutical composition, or it may be physically mixed with particles of a pharmaceutical composition. An excipient can be used, for example, to dilute an active agent and/or to modify properties of a pharmaceutical composition. Excipients can include, but are not limited to, antiadherents, binders, coatings, enteric coatings, disintegrants, flavorings, sweeteners, colorants, lubricants, glidants, sorbents, preservatives, adjuvants, carriers or vehicles. Excipients may be starches and modified starches, cellulose and cellulose derivatives, saccharides and their derivatives such as disaccharides, polysaccharides and sugar alcohols, protein, synthetic polymers, crosslinked polymers, antioxidants, amino acids or preservatives. Exemplary excipients include, but are not limited to, magnesium stearate, stearic acid, vegetable stearin, sucrose, lactose, starches, hydroxypropyl cellulose, hydroxypropyl methylcellulose, xylitol, sorbitol, maltitol, gelatin, polyvinylpyrrolidone (PVP), polyethyleneglycol (PEG), tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, sesame oil, propyl gallate, sodium metabisulphite or lanolin.

An “adjuvant” is an excipient that modifies the effect of other agents, typically the active ingredient. Adjuvants are often pharmacological and/or immunological agents. An adjuvant may modify the effect of an active ingredient by increasing an immune response. An adjuvant may also act as a stabilizing agent for a formulation. Exemplary adjuvants include, but are not limited to, aluminum hydroxide, alum, aluminum phosphate, killed bacteria, squalene, detergents, cytokines, paraffin oil, and combination adjuvants, such as freund's complete adjuvant or freund's incomplete adjuvant.

As used herein, “pharmaceutically acceptable salt” refers to pharmaceutically acceptable salts of a compound that are derived from a variety of organic and inorganic counter ions as will be known to a person of ordinary skill in the art and include, by way of example only, sodium, potassium, calcium, magnesium, ammonium, tetraalkylammonium, tris(hydroxymethyl)aminomethane (tris) and the like; and when the molecule contains a basic functionality, salts of organic or inorganic acids, such as hydrochloride, hydrobromide, tartrate, mesylate, benzene sulfonate, tosylate, succinate, acetate, maleate, oxalate, and the like. “Pharmaceutically acceptable acid addition salts” are a subset of “pharmaceutically acceptable salts” that retain the biological effectiveness of the free bases while formed by acid partners. In particular, the disclosed compounds form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzene sulfonic acid, isethionic acid, methanesulfonic acid, ethanesulfonic acid, p-toluenesulfonic acid, salicylic acid, xinafoic acid, gentisic acid, and the like. “Pharmaceutically acceptable base addition salts” are a subset of “pharmaceutically acceptable salts” that are derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, tris(hydroxymethyl)aminomethane (Tris), ethanolamine, 2-dimethylaminoethanol, 2-diethylaminoethanol, dicyclohexylamine, lysine (for example, L-lysine), arginine (for example, L-arginine), histidine, caffeine, procaine, hydrabamine, choline, betaine, ethylenediamine, glucosamine, meglumine, methylglucamine, theobromine, purines, piperazine, piperidine, N-ethylpiperidine, polyamine resins, and the like. Exemplary organic bases are isopropylamine, diethylamine, tris(hydroxymethyl)aminomethane (Tris), ethanolamine, trimethylamine, dicyclohexylamine, choline, and caffeine. (See, for example, S. M. Berge, et al., “Pharmaceutical Salts,” J. Pharm. Sci., 1977, 66:1-19 which is incorporated herein by reference.) In particular disclosed embodiments, the compound is in the form of a benzene sulfonate, hydrochloride, sodium, succinate, tris, mesylate, or tartrate salt.

“Pharmaceutically acceptable carrier” refers to an excipient that is a carrier or vehicle, such as a suspension aid, solubilizing aid, or aerosolization aid. Pharmaceutically acceptable carriers are conventional. Remington: The Science and Practice of Pharmacy, The University of the Sciences in Philadelphia, Editor, Lippincott, Williams, & Wilkins, Philadelphia. PA, 21^{st} Edition (2005), describes compositions and formulations suitable for pharmaceutical delivery of one or more therapeutic compositions and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

“Effective amount” with respect to a compound or composition refer to an amount of the compound or composition sufficient to achieve a particular desired result, such as to inhibit a protein or enzyme, particularly an interleukin-1 receptor-associated kinase; to elicit a desired biological or medical response in a tissue, system, subject or patient, to treat a specified disorder or disease; to ameliorate or eradicate one or more of its symptoms; and/or to prevent the occurrence of the disease or disorder. The amount of a compound which constitutes an “effective amount” may vary depending on the compound, the desired result, the disease state and its severity, the age of the patient to be treated, and the like.

“Prodrug” refers to compounds that are transformed in vivo to yield a biologically active compound, particularly the parent compound, for example, by hydrolysis in the gut or enzymatic conversion. Typically, a prodrug compound of a compound has less activity against a desired biological target that the parent compound. A prodrug negligible or less activity with respect to a desired target until it is metabolized to the active form. Common examples of prodrug moieties include, but are not limited to, ester, amide, carbamate, and urea forms of a compound having an active form bearing a carboxylic acid moiety. Examples of pharmaceutically acceptable esters of the compounds of this invention include, but are not limited to, esters of phosphate groups and carboxylic acids, such as aliphatic esters, particularly alkyl esters (for example C₁₋₆alkyl esters). Other prodrug moieties include phosphate esters, such as —(CH₂)ₙ—O—P(O)(OR′)₂, wherein n is 1 or 2 and R′ is H or C₁₋₆alkyl, such as —CH₂—O—P(O)(OR′)₂ wherein each R′ is independently H or C₁₋₆alkyl, when at least one R′ is H, the phosphate moiety can be in the form of a salt, such as a mono or dianion salt with an organic or inorganic cationic counter ion. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between one and six carbons). Amides and esters of disclosed exemplary embodiments of compounds according to the present invention can be prepared according to conventional methods. A thorough discussion of prodrugs is provided in T. Higuchi and V. Stella. “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. The compounds described herein can exist in un-solvated as well as solvated forms when combined with solvents, pharmaceutically acceptable or not, such as water, ethanol, and the like. Solvated forms of the presently disclosed compounds are within the scope of the embodiments disclosed herein.

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and as a consequence can exist as stereoisomers, such as double-bond isomers (i.e., geometric isomers), enantiomers, diastereomers, and mixtures thereof, such as racemic mixtures. As another example, certain disclosed compounds can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, it would be understood that the disclosed compounds encompass any tautomeric, conformational isomeric, optical isomeric, and/or geometric isomeric forms of the compounds described herein, as well as mixtures of these various different isomeric forms. In cases of limited rotation, e.g. around the amide bond or between two directly attached rings such as the pyrazole and pyridinyl rings, atropisomers are also possible and are also specifically included in the compounds of the invention.

Particular examples of the presently disclosed compounds include one or more asymmetric centers; thus these compounds can exist in different stereoisomeric forms. Accordingly, compounds and compositions may be provided as individual pure enantiomers or diastereomers, or as stereoisomeric mixtures, including racemic mixtures. In certain embodiments the compounds disclosed herein are synthesized in or are purified to be in substantially enantiopure form, such as in at least 90% enantiomeric excess, 95% enantiomeric excess, 97% enantiomeric excess, 98% enantiomeric excess, 99% enantiomeric excess, 99.5% enantiomeric excess, or greater than 99.5% enantiomeric excess, such as in enantiopure form.

In any embodiments, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, ethyl may be C₂H₅ or C₂H₅ where from 1 to hydrogens are replaced by deuterium.

### I. Compounds

The compound suitable for use in the methods provided herein is: 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate, which has the following structure:

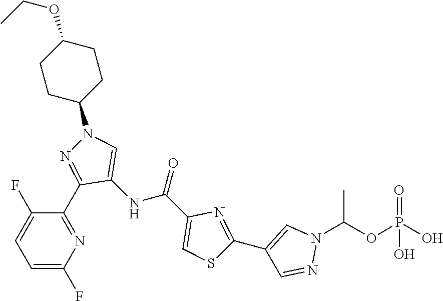 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]

or a stereoisomer or mixture of stereoisomers, a pharmaceutically acceptable salt, tautomer, solvate, hydrate, co-crystal, clathrate, or polymorph thereof (Compound 1).

In one embodiment, Compound 1 is an inorganic base salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, Compound 1 is a mono-inorganic base salt or a di-inorganic base salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, Compound 1 is an alkali metal salt, an alkaline earth metal salt, or an ammonium salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, Compound 1 is a mono-sodium salt, di-sodium salt, mono-potassium salt, di-potassium salt, calcium salt, magnesium salt, or mono-ammonium salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

In one embodiment, Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt (Compound IA):

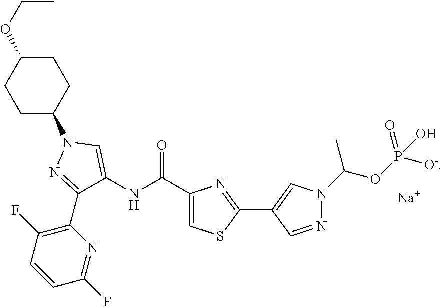

In one embodiment. Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate disodium salt (Compound 1B):

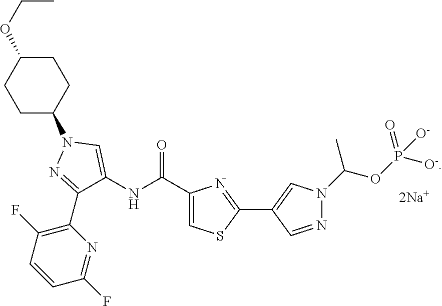

In one embodiment, Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate (Compound 1C):

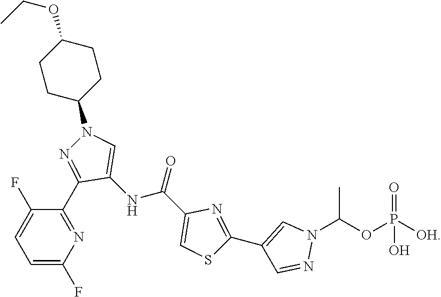 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]

Compound 1 can be prepared according to the methods described in the Examples provided herein or as described in U.S. Pat. No. 11,370,787 B2, the disclosure of which is incorporated herein by reference in its entirety. The compound can be also synthesized according to other methods apparent to those of skill in the art based upon the teaching herein.

### II. Methods of Use

In one embodiment, provided herein are methods for treating, preventing, managing, and/or ameliorating a disease by administering Compound 1 to a subject, wherein the disease is myelodysplastic syndrome (MDS), myeloproliferative neoplasms (MPN), MPN/MDS, clonal cytopenia of undetermined significance (CCUS), chronic myeloid leukemia (CML) or acute myeloid leukemia (AML). In one embodiment, provided herein are methods for treating, preventing, managing, and/or ameliorating a disease by administering Compound 1 to a subject, wherein the disease is myelodysplastic syndrome (MDS), myeloproliferative neoplasms (MPN), MPN/MDS, clonal cytopenia of undetermined significance (CCUS), chronic myeloid leukemia (CML), acute myeloid leukemia (AML), chronic myelomonocytic leukemia (CMML) or transfusion-dependent anemia due to myelodysplastic syndrome (MDS).

In one embodiment, Compound 1 is administered to the subject with food. In one embodiment, Compound 1 is administered to the subject without food.

In one embodiment, Compound 1 is administered to the subject with a low fat meal. In one embodiment, Compound 1 is administered to the subject with a meal that is not high in fat. In one embodiment, Compound 1 is administered to the subject with a high fat meal.

In one embodiment, Compound 1 is administered within 1 hour after a meal. In one embodiment, Compound 1 is administered within 45 minutes after a meal. In one embodiment, Compound 1 is administered within 30 minutes after a meal.

In one embodiment, Compound 1 is administered within 1 hour after a low fat meal. In one embodiment, Compound 1 is administered within 45 minutes after a low fat meal. In one embodiment, Compound 1 is administered within 30 minutes after a low fat meal.

In one embodiment. Compound 1 is administered at least 2 hours after a meal. In one embodiment, Compound 1 is administered at least 4 hours after a meal. In one embodiment, Compound 1 is administered at least 6 hours after a meal.

In one embodiment, Compound 1 is administered at least 2 hours before a meal. In one embodiment, Compound 1 is administered at least 4 hours before a meal. In one embodiment, Compound 1 is administered at least 6 hours before a meal.

In one embodiment, provided herein are methods for treating, managing, or ameliorating a disease comprising administering to a subject having the disease Compound 1, wherein Compound 1 is orally administered to the subject in an amount of about 100 mg to about 2000 mg per day, and the disease is myelodysplastic syndrome (MDS), myeloproliferative neoplasms (MPN), MPN/MDS, clonal cytopenia of undetermined significance (CCUS), chronic myeloid leukemia (CML) or acute myeloid leukemia (AML). In one embodiment, provided herein are methods for treating, managing, or ameliorating a disease comprising administering to a subject having the disease Compound 1, wherein Compound 1 is orally administered to the subject in an amount of about 100 mg to about 2000 mg per day, and the disease is myelodysplastic syndrome (MDS), myeloproliferative neoplasms (MPN), MPN/MDS, clonal cytopenia of undetermined significance (CCUS), chronic myeloid leukemia (CML), acute myeloid leukemia (AML), chronic myelomonocytic leukemia (CMML) or transfusion-dependent anemia is due to lower risk MDS. In one embodiment, provided herein are methods for treating, managing, or ameliorating a disease comprising administering to a subject having the disease Compound 1, wherein Compound 1 is orally administered to the subject in an amount of about 100 mg to about 1000 mg per day, and the disease is myelodysplastic syndrome (MDS), myeloproliferative neoplasms (MPN), MPN/MDS, clonal cytopenia of undetermined significance (CCUS), chronic myeloid leukemia (CML) or acute myeloid leukemia (AML).

In one embodiment, provided herein are methods for treating, managing, or ameliorating a myelodysplastic syndrome comprising administering to a subject having a myelodysplastic syndrome Compound 1, wherein Compound 1 is orally administered to the subject in an amount of about 100 mg to about 2000 mg per day.

In one embodiment, provided herein are methods for treating, preventing, managing, and/or ameliorating a myelodysplastic syndrome (MDS) by administering a therapeutically effective amount of Compound 1 to a subject. In one embodiment provided herein is a method for treating MDS. In one embodiment, the MDS is relapsed, resistant or refractory MDS. In one embodiment, MDS is refractory anemia (RA); RA with ringed sideroblasts (RARS); RA with excess of blasts (RAEB); refractory cytopenia with multilineage dysplasia (RCMD), refractory cytopenia with unilineage dysplasia (RCUD); unclassifiable myelodysplastic syndrome (MDS-U), myelodysplastic syndrome associated with an isolated del(5q) chromosome abnormality, therapy-related myeloid neoplasms or chronic myelomonocytic leukemia (CMML). In some embodiments, the MDS is very low risk, low risk, intermediate risk, high risk or very high risk MDS based on the Revised International Prognostic Scoring System (IPSS-R). In some embodiments, the MDS is very low, low, or intermediate-1 risk (IPSS-R≤3.5), collectively, lower-risk MDS (LR-MDS). In some embodiments, the MDS patient has bone marrow blast ≤5%. In one embodiment, the MDS is very low risk. In another embodiment, the MDS is low risk. In another embodiment, the MDS is intermediate risk. In another embodiment, the MDS is high risk. In another embodiment, the MDS is very high risk MDS. In some embodiments, the MDS is primary or de novo MDS. In other embodiments, the MDS is secondary MDS.

In yet certain embodiments, the MDS is transfusion dependent (TD) lower risk MDS (LR-MDS). In yet certain embodiments, the MDS is high transfusion burden MDS, wherein the patient requires about or greater than 8 red blood cell units for over 8 weeks or for over 16 weeks. In yet certain embodiments, the MDS is a low transfusion burden MDS, wherein the patient requires from about 3 to about 7 red blood cell units for over 8 weeks or for over 16 weeks. In yet certain embodiments, the MDS is transfusion dependent MDS wherein the patient requires about 4 or more red blood cell units over 8 weeks who have not responded to or have lost response to or are ineligible for erythropoiesis-stimulating agents (ESAs). In yet certain embodiments, the MDS is transfusion dependent MDS wherein the patient requires about 2 or more red blood cell units over 8 weeks. In yet certain embodiments, the MDS is MDS with symptomatic anemia with hemoglobin ≤9.0 g/dL and no red blood cell (RBC) transfusion for at least 16 weeks prior to administration of Compound 1. In yet certain embodiments, the MDS is transfusion dependent MDS, wherein the patient requires about or greater than 2 red blood cell units within 8 weeks in the preceding 16 weeks for a hemoglobin <9.0 g/dL prior to administration of Compound 1. In yet certain embodiments, the MDS is relapsed, refractory or ineligible for ESAs and have previously received one or more prior approved therapies for LR-MDS. In yet certain embodiments, the MDS is MDS with del (5q) mutation and which has failed prior lenalidomide therapy. In yet certain embodiments, the MDS is LR-MDS wherein the patient has transfusion-dependent anemia. In certain embodiments, the MDS is transfusion-dependent MDS which is relapsed, refractory to or have had inadequate response to prior therapies for MDS. In certain embodiments, the MDS is transfusion-dependent MDS which is relapsed, refractory to or have had inadequate response to prior therapies for MDS or to HMAs. In certain embodiments, the MDS is transfusion-dependent MDS which is relapsed, refractory to or have had inadequate response to hypomethylating agents. In yet certain embodiments, the MDS is ring sideroblast-negative (RS-negative MDS). In yet certain embodiments, the MDS is ring sideroblast-negative MDS wherein the patient is relapsed, refractor or ineligible for ESAs. In yet certain embodiments, the MDS is very low risk, low risk, moderate low risk, moderate high risk, high risk or very high risk MDS based on the Molecular International Prognostic scoring system IPSS-M. In one embodiment, MDS has a prognostic score of <=3 IPSS. In one embodiment, the MDS is relapsed, refractory to prior therapy such as luspatercept or imetelstat. In yet certain embodiments, the MDS is relapsed, refractory and/or eligible for prior therapy with ESAs. In yet certain embodiments, the MDS is relapsed or refractory to ESAs. In yet certain embodiments, the MDS is relapsed or refractory to HMAs. In yet certain embodiments, the MDS is relapsed or refractory to luspatercept or HMAs. In yet certain embodiments, the MDS is HMA naïve. In one embodiment, the MDS is previously-treated transfusion dependent LR-MDS.

In one embodiment provided herein is a method for treating transfusion-dependent anemia due to myelodysplastic syndrome (MDS). In another embodiment, the transfusion-dependent anemia is due to low- or intermediate-1-risk MDS. In another embodiment, the transfusion-dependent anemia is due to very low or low-risk MDS. In another embodiment, the transfusion-dependent anemia is due to very low, low, or intermediate-1 risk (IPSS-R≤3.5), collectively, lower-risk MDS (LR-MDS). In another embodiment, provided herein is a method for treating transfusion-dependent anemia due to myelodysplastic syndrome (MDS) associated with a deletion 5q abnormality with or without additional cytogenetic abnormalities. In another embodiment, provided herein is a method for treating transfusion-dependent anemia due to low- or intermediate-1-risk myelodysplastic syndrome (MDS) associated with a deletion 5q abnormality with or without additional cytogenetic abnormalities. In another embodiment, the transfusion-dependent anemia due to myelodysplastic syndrome (MDS) associated with a deletion 5q abnormality has failed lenalidomide therapy.

In one embodiment:
(i) the myelodysplastic syndrome is a higher-risk myelodysplastic or a lower-risk myelodysplastic syndrome;
(ii) the myelodysplastic syndrome is a refractory or relapsed lower-risk myelodysplastic syndrome;
(iii) the myelodysplastic syndrome is a relapsed, refractory/resistant, intolerant, or has an inadequate response to one or more therapies for myelodysplastic syndrome;
(iv) the myelodysplastic syndrome is a lower-risk myelodysplastic syndrome that is relapsed, refractory/resistant, intolerant, or has an inadequate response to one or more therapies for lower-risk myelodysplastic syndrome;
(v) the myelodysplastic syndrome is a transfusion-dependent lower-risk myelodysplastic syndrome;
(vi) the myelodysplastic syndrome is a transfusion-dependent lower-risk myelodysplastic syndrome that is non-responsive to or ineligible for treatment with erythropoiesis-stimulating agents;
(vii) the myelodysplastic syndrome is a lower-risk myelodysplastic syndrome that is relapsed, refractory to or ineligible for treatment with erythropoiesis-stimulating agents;
(viii) the myelodysplastic syndrome is a lower-risk myelodysplastic syndrome that is relapsed or refractory to erythropoiesis-stimulating agents;
(ix) the myelodysplastic syndrome is a lower-risk myelodysplastic syndrome that is relapsed, refractory to or ineligible for treatment with hypomethylating agents;
(x) the myelodysplastic syndrome is a lower-risk myelodysplastic syndrome that is relapsed or refractory to hypomethylating agents;
(xi) the myelodysplastic syndrome is a lower-risk myelodysplastic syndrome that is relapsed, refractory to or ineligible for treatment with erythropoiesis-stimulating agents and has previously been treated with one or more therapies for lower-risk myelodysplastic syndrome;
(xii) the myelodysplastic syndrome is a transfusion-dependent lower-risk myelodysplastic syndrome that is relapsed or refractory to erythropoiesis-stimulating agents;
(xiii) the myelodysplastic syndrome is a transfusion-dependent lower-risk myelodysplastic syndrome that is relapsed or refractory to hypomethylating agents;
(xiv) the myelodysplastic syndrome is a transfusion-dependent lower-risk myelodysplastic syndrome that is relapsed, refractory to or ineligible for treatment with erythropoiesis-stimulating agents and has been previously received with one or more therapies for lower-risk myelodysplastic syndrome;
(xv) the myelodysplastic syndrome is ring sideroblast-negative; or
(xvi) the myelodysplastic syndrome is with del (5q) mutation and failed prior lenalidomide therapy.

In one embodiment:
(i) the myelodysplastic syndrome is a higher-risk myelodysplastic or a lower-risk myelodysplastic syndrome;
(ii) the myelodysplastic syndrome is a refractory or relapsed lower-risk myelodysplastic syndrome;
(iii) the myelodysplastic syndrome is a relapsed, refractory/resistant, intolerant, or has an inadequate response to one or more therapies for myelodysplastic syndrome;
(iv) the myelodysplastic syndrome is a lower-risk myelodysplastic syndrome that is relapsed, refractory/resistant, intolerant, or has an inadequate response to one or more therapies for lower-risk myelodysplastic syndrome;
(v) the myelodysplastic syndrome is a transfusion-dependent lower-risk myelodysplastic syndrome;
(vi) the myelodysplastic syndrome is a lower-risk myelodysplastic syndrome that is relapsed or refractory to erythropoiesis-stimulating agents;
(vii) the myelodysplastic syndrome is a lower-risk myelodysplastic syndrome that is relapsed or refractory to hypomethylating agents;
(viii) the myelodysplastic syndrome is a lower-risk myelodysplastic syndrome that is relapsed or refractory to treatment with erythropoiesis-stimulating agents and has previously been treated with one or more therapies for lower-risk myelodysplastic syndrome;
(ix) the myelodysplastic syndrome is a transfusion-dependent lower-risk myelodysplastic syndrome that is relapsed or refractory to erythropoiesis-stimulating agents;
(x) the myelodysplastic syndrome is a transfusion-dependent lower-risk myelodysplastic syndrome that is relapsed or refractory to hypomethylating agents;
(xi) the myelodysplastic syndrome is a transfusion-dependent lower-risk myelodysplastic syndrome that is relapsed, refractory to or ineligible for treatment with erythropoiesis-stimulating agents and has been previously received with one or more therapies for lower-risk myelodysplastic syndrome;
(xii) the myelodysplastic syndrome is ring sideroblast-negative; or
(xiii) the myelodysplastic syndrome is with del (5q) mutation and failed prior lenalidomide therapy.

In another embodiment, the prior therapies for myelodysplastic syndrome or lower myelodysplastic syndrome comprises a treatment with an erythropoiesis-stimulating agent (ESA), erythroid maturation agent (EMA), telomerase inhibitor, immunomodulatory drug (IMiD), granulocyte colony-stimulating factor (G-CSF) or a hypomethylating agent, or a combination thereof. In another embodiment, the prior therapies for myelodysplastic syndrome or lower myelodysplastic syndrome comprises a treatment with an ESA, luspatercept, imetelstat, lenalidomide, granulocyte colony-stimulating factor (G-CSF) or a hypomethylating agent, or a combination thereof. In another embodiment, the myelodysplastic syndrome has relapsed or progressed after one or more prior therapies. In another embodiment, the myelodysplastic syndrome has relapsed or progressed after one prior therapy. In another embodiment, the myelodysplastic syndrome has relapsed or progressed after one or more prior therapies of an ESA. In another embodiment, the myelodysplastic syndrome has relapsed or progressed after one prior therapy of an ESA. In another embodiment, the myelodysplastic syndrome has relapsed or progressed after one or more prior therapies of an HMA. In another embodiment, the myelodysplastic syndrome has relapsed or progressed after one prior therapy of an HMA. In another embodiment, the myelodysplastic syndrome has relapsed or progressed after one or more prior therapies of an ESA or HMA. In another embodiment, the myelodysplastic syndrome has relapsed or progressed after one prior therapy of an ESA or HMA. In another embodiment, the myelodysplastic syndrome has relapsed or progressed after two prior therapies one of which included an ESA. In another embodiment, the myelodysplastic syndrome has relapsed or progressed after three prior therapies one of which included an ESA. In another embodiment, the myelodysplastic syndrome has relapsed or progressed after two prior therapies one of which included an HMA. In another embodiment, the myelodysplastic syndrome has relapsed or progressed after three prior therapies one of which included an HMA. In yet another embodiment, the myelodysplastic syndrome has relapsed or progressed after two prior therapies one of which included an ESA or HMA. In yet another embodiment, the myelodysplastic syndrome has relapsed or progressed after three prior therapies one of which included an ESA or HMA. Further provided herein are methods for achieving one or more clinical endpoints associated with MDS comprising administering a therapeutically effective amount of Compound 1 to a patient in need thereof.

In certain embodiments, the methods provided herein increase the overall survival (OS), complete remission rate (CRR), objective response rate (ORR), time to progression, relapse free survival (RFS), progression-free survival (PFS) event-free survival, duration of remission, duration of response, and/or time to remission/response in a patient population having MDS treated with an effective amount of Compound 1, when compared to a patient population not treated with Compound 1.

In one embodiment, the methods provided herein increase the overall survival (OS) in a patient population having MDS treated with an effective amount of Compound 1, when compared to a patient population not treated with Compound 1.

In one embodiment, the methods provided herein increase the complete remission rate (CRR) in a patient population having MDS treated with an effective amount of Compound 1, when compared to a patient population not treated with Compound 1.

In one embodiment, the methods provided herein increase the objective response rate (ORR) in a patient population having MDS treated with an effective amount of Compound 1, when compared to a patient population not treated with Compound 1.

In one embodiment, the methods provided herein increase the time to progression in a patient population having MDS treated with an effective amount of Compound 1, when compared to a patient population not treated with Compound 1.

In one embodiment, the methods provided herein increase the relapse free survival (RFS) in a patient population having MDS treated with an effective amount of Compound 1, when compared to a patient population not treated with Compound 1.

In one embodiment, the methods provided herein increase the progression-free survival (PFS) in a patient population having MDS treated with an effective amount of Compound 1, when compared to a patient population not treated with Compound 1.

In one embodiment, the methods provided herein increase the event-free survival in a patient population having MDS treated with an effective amount of Compound 1, when compared to a patient population not treated with Compound 1.

In one embodiment, the methods provided herein increase the duration of remission in a patient population having MDS treated with an effective amount of Compound 1, when compared to a patient population not treated with Compound 1.

In one embodiment, the methods provided herein increase the duration of response in a patient population having MDS treated with an effective amount of Compound 1, when compared to a patient population not treated with Compound 1.

In one embodiment, the methods provided herein increase the time to remission/response in a patient population having MDS treated with an effective amount of Compound 1, when compared to a patient population not treated with Compound 1.

In certain embodiment, the ORR includes all responses of complete remission (CR) (i.e., morphologic leukemia-free state, morphologic CR, cytogenetic CR, molecular CR, and morphologic CR with incomplete blood recovery), and partial remission.

In one embodiment, the methods provided herein result in red blood cell (RBC) transfusion independence in a patient population having MDS treated with an effective amount of Compound 1, when compared to a patient population not treated with Compound 1. In yet certain embodiments, the methods provided herein result in red blood cell (RBC) transfusion independence of about equal to or greater than 8 weeks according to the IWG 2006 criteria. In yet certain embodiments, the methods provided herein result in red blood cell (RBC) transfusion independence of more than 16 weeks or more than 24 weeks. In yet certain embodiments, the methods provided herein result in reduced transfusion dependence from baseline, or hematologic improvement-erythroid (HI-E) per IWG 2018 criteria. In yet certain embodiments, the methods provided herein result in improved patient reported outcomes, for example, as measured by the Functional Assessment of Chronic Illness Therapy (FACIT) Fatigue Scale.

In one embodiment, provided herein are methods for treating, preventing, managing, and/or ameliorating myeloproliferative neoplasms (MPN) by administering Compound 1 to a subject.

In one embodiment, provided herein are methods for treating, preventing, managing, and/or ameliorating MPN/MDS by administering Compound 1 to a subject.

In one embodiment, provided herein are methods for treating, preventing, managing, and/or ameliorating clonal cytopenia of undetermined significance (CCUS) by administering Compound 1 to a subject.

In one embodiment, provided herein are methods for treating, preventing, managing, and/or ameliorating chronic myeloid leukemia (CML) by administering Compound 1 to a subject.

### III. Dosages/Dosing Regimens

In one embodiment, provided herein are methods for treating, preventing, managing, and/or ameliorating a disease by administering Compound 1 to a subject in an amount of about 100 mg per day to about 2000 mg per day. In one embodiment, the methods comprise administering Compound 1 in an amount of about 10 mg per day to about 1000 mg per day. In one embodiment, the methods comprise administering Compound 1 in an amount of about 200 mg per day to about 850 mg per day. In one embodiment, the methods comprise administering Compound 1 in an amount of about 100 mg per day, about 125 mg per day, about 150 mg per day, about 200 mg per day, about 250 mg per day, about 300 mg per day, about 350 mg per day, about 375 mg per day, about 400 mg per day, about 450 mg per day, about 500 mg per day, about 550 mg per day, about 600 mg per day, about 650 mg per day, about 700 mg per day, about 750 mg per day, about 800 mg per day, about 850 mg per day, about 900 mg per day, about 950 mg per day or about 1000 mg per day. In certain embodiments, Compound 1 is administered in these amounts in one, two, three or four doses per day.

In one embodiment, the methods comprise administering Compound 1 in an amount of about 125 mg per day, about 150 mg per day, about 200 mg per day, about 250 mg per day, about 300 mg per day, about 375 mg per day, about 400 mg per day, about 500 mg per day, about 750 mg per day or about 1000 mg per day. In certain embodiments, Compound 1 is administered in these amounts in one, two, three or four doses per day.

In one embodiment, the methods comprise administering Compound 1 in an amount of about 125 mg per day. In one embodiment, the methods comprise administering Compound 1 in an amount of about 150 mg per day. In one embodiment, the methods comprise administering Compound 1 in an amount of about 250 mg per day. In one embodiment, the methods comprise administering Compound 1 in an amount of about 300 mg per day. In one embodiment, the methods comprise administering Compound 1 in an amount of about 375 mg per day. In one embodiment, the methods comprise administering Compound 1 in an amount of about 400 mg per day. In one embodiment, the methods comprise administering Compound 1 in an amount of about 500 mg per day. In one embodiment, the methods comprise administering Compound 1 in an amount of about 750 mg per day. In one embodiment, the methods comprise administering Compound 1 in an amount of about 800 mg per day. In one embodiment, the methods comprise administering Compound 1 in an amount of about 1000 mg per day. In certain embodiments, Compound 1 is administered in these amounts in one, two, three or four doses per day.

In one embodiment, the methods comprise administering Compound 1 in an amount of about 150 mg twice daily. In one embodiment, the methods comprise administering Compound 1 in an amount of about 200 mg twice daily. In one embodiment, the methods comprise administering Compound 1 in an amount of about 250 mg twice daily. In one embodiment, the methods comprise administering Compound 1 in an amount of about 375 mg twice daily. In one embodiment, the methods comprise administering Compound 1 in an amount of about 400 mg twice daily. In one embodiment, the methods comprise administering Compound 1 in an amount of about 500 mg twice daily.

In one embodiment, the methods comprise administering Compound 1 in an amount of about 300 mg per day, in two doses of about 150 mg each. In one embodiment, the methods comprise administering Compound 1 in an amount of about 500 mg per day, in two doses of about 250 mg each. In one embodiment, the methods comprise administering Compound 1 in an amount of about 750 mg per day, in two doses of about 375 mg each. In one embodiment, the methods comprise administering Compound 1 in an amount of about 800 mg per day, in two doses of about 400 mg each. In one embodiment, the methods comprise administering Compound 1 in an amount of about 1000 mg per day, in two doses of about 500 mg each.

In one embodiment, the methods comprise administering Compound 1 in an amount of about 750 mg per day in two doses, wherein a first dose is about 500 mg and a second dose is about 250 mg.

In one embodiment, the methods comprise administering Compound 1 in an amount of about 750 mg per day in two doses, wherein a first dose is about 250 mg and a second dose is about 500 mg.

In one embodiment, the first dose and the second dose of Compound 1 are administered at an interval of about 6-15 hours. In one embodiment, the first dose and the second dose are administered at an interval of about 6, 8, 10, 12 or 15 hours.

In one embodiment, the methods comprise administering Compound 1 in an amount corresponding to about 100 mg per day to about 200 mg per day, about 200 mg per day to about 300 mg per day, about 450 mg per day to about 550 mg per day, about 750 mg per day to about 850 mg per day or about 950 mg per day to about 1000 mg per day of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

In one embodiment, the methods comprise administering Compound 1 in an amount corresponding to about 250 mg per day, about 500 mg per day, about 750 mg per day, about 125 mg twice daily, about 250 mg twice daily, about 400 mg twice daily, about 500 mg twice daily, about 250 mg once daily, about 500 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

In one embodiment, the methods comprise administering Compound 1 in an amount corresponding to about 250 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 400 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 500 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 750 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 1000 mg once daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

In one embodiment, the methods comprise administering Compound 1 in an amount corresponding to about 150 mg twice daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 250 mg twice daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 400 mg twice daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate. In one embodiment, the methods comprise administering Compound 1 in an amount corresponding to about 500 mg twice daily of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

In certain embodiments of the methods provided herein, the patient is relapsed, refractory/resistant, intolerant, or has an inadequate response to one or more prior therapies for myelodysplastic syndrome. In certain embodiments of the methods provided herein, the patient is relapsed, refractory/resistant, intolerant, or has an inadequate response to one or more prior therapies for myelodysplastic syndrome. In certain embodiments, prior therapies for myelodysplastic syndromes are described in National Comprehensive Cancer Network (NCCN) Guidelines for Myelodysplastic Syndromes, for example NCCN Guidelines Version 2.2025 Myelodysplastic Syndromes. In one embodiment, the prior therapy comprises a treatment with erythropoiesis-stimulating agents (ESA) or a hypomethylating agent or a combination thereof. In another embodiment, the prior therapy comprises a treatment with an ESA, luspatercept or a hypomethylating agent. In another embodiment, the prior therapy comprises a treatment with an ESA, luspatercept, imetelstat, lenalidomide, granulocyte colony-stimulating factor (G-CSF) or a hypomethylating agent, or a combination thereof. In another embodiment, the prior therapy comprises a treatment with an ESA, luspatercept, lenalidomide or a hypomethylating agent, or a combination thereof. In another embodiment, the prior therapy comprises a treatment with an ESA, IL-1β inhibitor, SYK inhibitor, luspatercept, imetelstat, lenalidomide, granulocyte colony-stimulating factor (G-CSF), mIDH1-inhibitor, or a hypomethylating agent, or a combination thereof. In another embodiment, the prior therapy comprises a treatment with an ESA, IL-1β inhibitor, ALK2 inhibitor, SYK inhibitor, luspatercept, imetelstat, lenalidomide, granulocyte colony-stimulating factor (G-CSF), mIDH1-inhibitor, anti-TIM-3 antibody or a hypomethylating agent, or a combination thereof. In another embodiment, the prior therapy comprises a treatment with an ESA, canakinumab, luspatercept, imetelstat, lenalidomide, granulocyte colony-stimulating factor (G-CSF), ivosidenib, olutasidenib or a hypomethylating agent, or a combination thereof. In another embodiment, the prior therapy comprises a treatment with an ESA, luspatercept, imetelstat, lenalidomide, granulocyte colony-stimulating factor (G-CSF), mIDH1-inhibitor or a hypomethylating agent, or a combination thereof. In one embodiment, the erythropoiesis-stimulating agents (ESA) is erythropoietin (EPO). In one embodiment, the erythropoiesis-stimulating agents (ESA) is erythropoietin (EPO), epoetin, darbepoetin or methoxy polyethylene glycol-epoetin s. In another embodiment, the erythropoiesis-stimulating agents (ESA) is erythropoietin (EPO), epoetin alpha or darbepoetin alfa. In another embodiment, the erythropoiesis-stimulating agents (ESA) is epoetin, darbepoetin or methoxy polyethylene glycol-epoetin β. In yet another embodiment, the erythropoiesis-stimulating agents (ESA) is epoetin alpha or darbepoetin alfa. In one embodiment, the prior therapy comprises a treatment with an erythropoietin, luspatercept, or a hypomethylating agent. In one embodiment, the prior therapy comprises treatment with a hypomethylating agent. In one embodiment, the hypomethylating agent is azacytidine or decitabine. In one embodiment, the hypomethylating agent is azacytidine, decitabine or a combination of decitabine and cedazuridine. In certain embodiments, azacytidine or decitabine is administered with venetoclax. In certain embodiments, the prior therapy is ivosidenib or olutasidenib if the myelodysplastic syndrome has the IDH1 mutation. In certain embodiments, the methods provided herein are for treatment of patients who have failed prior lenalidomide therapy. In certain embodiments of the methods provided herein, the patient has myelodysplastic syndrome associated with an isolated del(5q) chromosome abnormality who has failed prior lenalidomide therapy.

In certain embodiments, the methods provided herein are for treatment of patients 18 years or older.

### IV. Combination Therapy

In certain embodiments, the methods provided herein comprise administering a therapeutically effective amount of Compound 1 in combination with one another, or as an adjunct to, or in combination with, other established therapies. In another aspect, the methods comprise administering Compound 1 in combination with other therapeutic agents useful for the disorder or condition being treated. These compounds may be administered simultaneously, sequentially in any order, by the same route of administration, or by a different route.

In some embodiments, the methods provided herein comprise administering a therapeutically effective amount of Compound 1 with another therapeutic agent, such as an analgesic, an antibiotic, an anticoagulant, an antibody, an anti-inflammatory agent, an immunosuppressant, a guanylate cyclase-C agonist, an intestinal secretagogue, an antiviral, anticancer, antifungal, or a combination thereof. In certain embodiments, the second therapeutic is an anti-inflammatory agent, an immunosuppressant and/or may be a steroid. In certain embodiments, the second therapeutic is a hypomethylating agent, an immunomodulatory imide drug or a mutant IDH1 inhibitor.

These various agents can be used in accordance with their standard or common dosages, as specified in the prescribing information accompanying commercially available forms of the drugs (see also, the prescribing information in the 2006 Edition of The Physician's Desk Reference), the disclosures of which are incorporated herein by reference.

### V. Compositions Comprising Compound 1

In one embodiment, the methods provided herein comprise administering Compound 1 alone, in any combination with, or adjunctive to, at least one second therapeutic agent, and further Compound 1, and the at least one second therapeutic if present, may be used in combination with any suitable additive useful for forming compositions for administration to a subject. Additives can be included in pharmaceutical compositions for a variety of purposes, such as to dilute a composition for delivery to a subject, to facilitate processing of the formulation, to provide advantageous material properties to the formulation, to facilitate dispersion from a delivery device, to stabilize the formulation (e.g., antioxidants or buffers), to provide a pleasant or palatable taste or consistency to the formulation, or the like. Typical additives include, by way of example and without limitation: pharmaceutically acceptable excipients; pharmaceutically acceptable carriers; and/or adjuvants, such as mono-, di-, and polysaccharides, sugar alcohols and other polyols, such as, lactose, glucose, raffinose, melezitose, lactitol, maltitol, trehalose, sucrose, mannitol, starch, or combinations thereof; surfactants, such as sorbitols, diphosphatidyl choline, and lecithin; bulking agents; buffers, such as phosphate and citrate buffers; anti-adherents, such as magnesium stearate; binders, such as saccharides (including disaccharides, such as sucrose and lactose), polysaccharides (such as starches, cellulose, microcrystalline cellulose, cellulose ethers (such as hydroxypropyl cellulose), gelatin, synthetic polymers (such as polyvinylpyrrolidone, polyalkylene glycols); coatings (such as cellulose ethers, including hydroxypropylmethyl cellulose, shellac, corn protein zein, and gelatin); release aids (such as enteric coatings); disintegrants (such as crospovidone, crosslinked sodium carboxymethyl cellulose, and sodium starch glycolate); fillers (such as dibasic calcium phosphate, vegetable fats and oils, lactose, sucrose, glucose, mannitol, sorbitol, calcium carbonate, and magnesium stearate); flavors and sweeteners (such as mint, cherry, anise, peach, apricot or licorice, raspberry, and vanilla; lubricants (such as minerals, exemplified by talc or silica, fats, exemplified by vegetable stearin, magnesium stearate or stearic acid); preservatives (such as antioxidants exemplified by vitamin A, vitamin E, vitamin C, retinyl palmitate, and selenium, amino acids, exemplified by cysteine and methionine, citric acid and sodium citrate, parabens, exemplified by methyl paraben and propyl paraben); colorants; compression aids; emulsifying agents; encapsulation agents; gums; granulation agents; and combinations thereof.

In certain embodiments, the methods provided herein comprise administering a pharmaceutical composition comprising:
a) about 15% to about 35% Compound 1 by weight based on total weight of the tablet;
b) about 50% to about 70% intra-granular excipient by weight based on total weight of the tablet; and
c) about 10% to about 20% extra-granular excipient by weight based on total weight of the pharmaceutical composition.

In certain embodiments, the pharmaceutical composition comprises 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate disodium salt.

In certain embodiments, the pharmaceutical composition comprises 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

In certain embodiments, the pharmaceutical composition comprises 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

In certain embodiments, the pharmaceutical compositions comprise: Compound 1, present in an amount from about 20% to about 30% by weight based on total weight of the pharmaceutical composition; intra-granular excipients from about 55% to about 65% by weight based on total weight of the pharmaceutical composition, wherein the intra-granular excipients comprise one or more of a diluent, a disintegrant, a glidant and a lubricant; and extra-granular excipients from about 10% to about 15% by weight based on total weight of the pharmaceutical composition, wherein the extra-granular excipients comprise one or more of a compression aid, a disintegrant, a glidant and a lubricant.

In certain embodiments, the pharmaceutical compositions comprise: Compound 1, present in an amount from about 20% to about 30% by weight based on total weight of the pharmaceutical composition; intra-granular excipients from about 55% to about 65% by weight based on total weight of the pharmaceutical composition, wherein the intra-granular excipients comprise one or more of microcrystalline cellulose, mannitol, croscarmellose sodium, fumed silica, and sodium stearyl fumarate; and extra-granular excipients from about 10% to about 15% by weight based on total weight of the pharmaceutical composition, wherein the extra-granular excipients comprise one or more of microcrystalline cellulose, croscarmellose sodium, and sodium stearyl fumarate.

In certain embodiments, the pharmaceutical composition comprises from about 20% to about 30% Compound 1 by weight based on total weight of the pharmaceutical composition. In certain embodiments, the pharmaceutical composition comprises about 20%, about 22%, about 24%, about 25%, about 25.67%, about 27%, or about 30% Compound 1 by weight based on total weight of the pharmaceutical composition. In certain embodiments, the pharmaceutical composition comprises about 25.67% or about 25% Compound 1 by weight based on total weight of the pharmaceutical composition.

In certain embodiments, the pharmaceutical composition comprises about 60%, about 61%, about 62%, about 62.5%, about 63%, about 64%, about 65% or about 70% intra-granular excipients by weight based on total weight of the pharmaceutical composition.

In certain embodiments, the intra-granular excipients comprise a diluent, a disintegrant, a glidant and a lubricant. In certain embodiments, the pharmaceutical composition comprises a diluent comprising microcrystalline cellulose. In certain embodiments, the intragranular excipients in the tablet comprise a diluent in an amount of about 55% to about 65% by weight based on the total weight of the tablet. In certain embodiments, the intragranular excipients in the tablet comprise a diluent in an amount of about 57% to about 63% by weight based on the total weight of the tablet. In certain embodiments, the intragranular excipients in the tablet comprise a diluent in an amount of about 57% to about 58% by weight based on the total weight of the tablet. In certain embodiments, the intragranular excipients in the tablet comprise a diluent in an amount of about 58% to about 59% by weight based on the total weight of the tablet. In certain embodiments, the intragranular excipients in the tablet comprise a diluent in an amount of about 55%, about 56%, about 57%, about 57.8%, about 58%, about 58.5%, about 59%, about 60%, about 62%, about 63%, or about 65% by weight based on the total weight of the tablet. In certain embodiments, the intragranular excipients in the tablet comprise a diluent in an amount of about 57%, about 57.8%, about 58%, about 58.5% or about 59% by weight based on the total weight of the tablet.

In one embodiment, the diluent in the tablet provided herein comprises microcrystalline cellulose and mannitol. In certain embodiments, the intragranular excipients in the tablet comprise microcrystalline cellulose in an amount of about 25% to about 35% by weight based on the total weight of the tablet, such as for example about 27% to about 33% by weight based on the total weight of the tablet. In certain embodiments, the intragranular excipients in the tablet comprise microcrystalline cellulose in an amount of about 28%, about 28.92%, about 29%, about 29.25%, about 30% or about 32% by weight based on the total weight of the tablet. In certain embodiments, the intragranular excipients in the tablet comprise microcrystalline cellulose in an amount of about 28.92% by weight based on the total weight of the tablet. In certain embodiments, the intragranular excipients in the tablet comprise microcrystalline cellulose in an amount of about 29.25% by weight based on the total weight of the tablet. In certain embodiments, the intragranular excipients in the tablet comprise mannitol in an amount of about 25% to about 35% by weight based on the total weight of the tablet, such as for example about 27% to about 33% by weight based on the total weight of the tablet. In certain embodiments, the intragranular excipients in the tablet comprise mannitol in an amount of about 28%, about 28.92%, about 29%, about 29.25%, about 30% or about 32% by weight based on the total weight of the tablet. In certain embodiments, the intragranular excipients in the tablet comprise mannitol in an amount of about 28.92% by weight based on the total weight of the tablet. In certain embodiments, the intragranular excipients in the tablet comprise mannitol in an amount of about 29.25% by weight based on the total weight of the tablet. In certain embodiments, the intragranular excipients in the tablet comprise microcrystalline cellulose and mannitol each in an amount of about 25% to about 35% by weight based on the total weight of the tablet, such as for example about 27% to about 33% by weight based on the total weight of the tablet. In certain embodiments, the intragranular excipients in the tablet comprise microcrystalline cellulose and mannitol each in an amount of about 28%, about 28.92%, about 29%, about 29.25%, about 30% or about 32% by weight based on the total weight of the tablet. In certain embodiments, the intragranular excipients in the tablet comprise microcrystalline cellulose and mannitol each in an amount of about 28.92% by weight based on the total weight of the tablet. In certain embodiments, the intragranular excipients in the tablet comprise microcrystalline cellulose and mannitol each in an amount of about 29.25% by weight based on the total weight of the tablet.

In one embodiment, the intragranular excipients in the tablet comprise a disintegrant in about 2% to about 4% by weight based on the total weight of the tablet. In one embodiment, the tablet comprises a disintegrant in about 2.5%, about 3% or about 4% by weight based on the total weight of the tablet. In one embodiment, the intragranular excipients in the tablet comprise a disintegrant in about 3% by weight based on the total weight of the tablet. In one embodiment, the disintegrant in the tablet provided herein is croscarmellose sodium. In one embodiment, the intragranular excipients in the tablet comprise croscarmellose sodium in the amount of about 2% to about 4% by weight based on the total weight of the tablet. In one embodiment, the intragranular excipients in the tablet comprise croscarmellose sodium in the amount of about 2.5%, about 3% or about 4% by weight based on the total weight of the tablet. In one embodiment, the intragranular excipients in the tablet comprise croscarmellose sodium in the amount of about 3% by weight based on the total weight of the tablet.

In one embodiment, the glidant used in the tablets provided herein is fumed silica or colloidal silicon dioxide. In one embodiment, the glidant used in the tablets provided herein is fumed silica. In certain embodiments, the intragranular excipients in the tablet comprise a glidant in an amount of about 0.2% to about 2% by weight based on the total weight of the tablet. In certain embodiments, the intragranular excipients in the tablet comprise fumed silica which is present in an amount of about 0.5%, about 1% or about 2% by weight based on the total weight of the tablet. In certain embodiments, the intragranular excipients in the tablet comprise fumed silica which is present in an amount of about 1% by weight based on the total weight of the tablet. In certain embodiments, the intragranular excipients in the tablet comprise fumed silica which is present in an amount of about 0.5% by weight based on the total weight of the tablet.

In certain embodiments, the pharmaceutical composition comprises a lubricant comprising sodium stearyl fumarate. In one embodiment, the intragranular excipients in the tablet comprise sodium stearyl fumarate in an amount of about 0.5% to about 2% by weight based on the total weight of the tablet. In one embodiment, the intragranular excipients in the tablet comprise sodium stearyl fumarate in an amount of about 0.5%, about 1% or about 2% by weight based on the total weight of the tablet. In one embodiment, the intragranular excipients in the tablet comprise sodium stearyl fumarate in an amount of about 1% by weight based on the total weight of the tablet.

In certain embodiments, the pharmaceutical composition comprises an extra-granular excipient comprising from a compression aid, a disintegrant, a glidant and a lubricant. In one embodiment, the extra-granular excipient comprises a compression aid comprising microcrystalline cellulose. In certain embodiments, the compression aid in the extragranular excipient is microcrystalline cellulose. In certain embodiments, the extragranular excipients in the tablet comprise microcrystalline cellulose in an amount of about 6% to about 10% by weight based on the total weight of the tablet. In certain embodiments, the extragranular excipients in the tablet comprise microcrystalline cellulose in an amount of about 6%, about 8% or about 10% by weight based on the total weight of the tablet. In certain embodiments, the extragranular excipients in the tablet comprise microcrystalline cellulose in an amount of about 7% by weight based on the total weight of the tablet. In certain embodiments, the extragranular excipients in the tablet comprise microcrystalline cellulose in an amount of about 8% by weight based on the total weight of the tablet.

In one embodiment, the disintegrant used in the extragranular excipient is croscarmellose sodium. In certain embodiments, the extragranular excipients in the tablet comprise croscarmellose sodium in an amount of about 2% to about 4% by weight based on the total weight of the tablet. In certain embodiments, the extragranular excipients in the tablet comprise croscarmellose sodium in an amount of about 2.5%, about 3% or about 4% by weight based on the total weight of the tablet. In certain embodiments, the extragranular excipients in the tablet comprise croscarmellose sodium in an amount of about 3% by weight based on the total weight of the tablet.

In one embodiment, the lubricant used in the extragranular excipient is sodium stearyl fumarate. In certain embodiments, the extragranular excipients in the tablet comprise sodium stearyl fumarate in an amount of about 1% to about 3% by weight based on the total weight of the tablet. In certain embodiments, the extragranular excipients in the tablet comprise sodium stearyl fumarate in an amount of about 1%, about 2% or about 3% by weight based on the total weight of the tablet. In certain embodiments, the extragranular excipients in the tablet comprise sodium stearyl fumarate in an amount of about 2% by weight based on the total weight of the tablet.

In one embodiment, the tablet provided herein comprises:
a) about 20% to about 30% Compound 1 by weight based on total weight of the tablet;
b) an intra-granular excipient comprising about 25% to about 35% microcrystalline cellulose by weight based on total weight of the tablet, about 25% to about 35% mannitol by weight based on total weight of the tablet, about 2% to about 4% croscarmellose sodium by weight based on total weight of the tablet, about 0.2% to about 2% fumed silica by weight based on total weight of the tablet, and about 0.5% to about 2% sodium stearyl fumarate by weight based on total weight of the tablet; and
c) an extra-granular excipient comprising about 6% to about 10% microcrystalline cellulose by weight based on total weight of the tablet, about 2% to about 4% croscarmellose sodium by weight based on total weight of the tablet, and about 1% to about 3% sodium stearyl fumarate by weight based on total weight of the tablet.

In one embodiment, the tablets provided herein having the desired stability during storage include a tablet comprising:
a) about 25% Compound 1 by weight based on total weight of the tablet;
b) the intra-granular excipient comprising about 29% microcrystalline cellulose by weight based on total weight of the tablet, about 29% mannitol by weight based on total weight of the tablet, about 3% croscarmellose sodium by weight based on total weight of the tablet, about 0.5% fumed silica by weight based on total weight of the tablet, and about 1% sodium stearyl fumarate by weight based on total weight of the tablet; and
c) the extra-granular excipient comprising about 7% microcrystalline cellulose by weight based on total weight of the tablet, about 3% croscarmellose sodium by weight based on total weight of the tablet, and about 2% sodium stearyl fumarate by weight based on total weight of the tablet.

In one embodiment, the tablets provided herein having the desired stability during storage include a tablet comprising:
a) about 25.7% Compound 1 by weight based on total weight of the tablet;
b) the intra-granular excipient comprising about 29% microcrystalline cellulose by weight based on total weight of the tablet, about 29% mannitol by weight based on total weight of the tablet, about 3% croscarmellose sodium by weight based on total weight of the tablet, about 0.5% fumed silica by weight based on total weight of the tablet, and about 1% sodium stearyl fumarate by weight based on total weight of the tablet; and
c) the extra-granular excipient comprising about 7% microcrystalline cellulose by weight based on total weight of the tablet, about 3% croscarmellose sodium by weight based on total weight of the tablet, and about 2% sodium stearyl fumarate by weight based on total weight of the tablet.

In one embodiment, Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium. In one embodiment, Compound 1 is solid Form E of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium.

In one embodiment, the tablet comprises:
a) about 250.00 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) intra-granular excipients comprising about 292.50 mg microcrystalline cellulose, about 292.50 mg mannitol, about 30 mg croscarmellose sodium, about 5 mg fumed silica, and about 10 mg sodium stearyl fumarate; and
c) the extra-granular excipient comprising 70 mg microcrystalline cellulose, about 30 mg croscarmellose sodium, and about 20 mg sodium stearyl fumarate.

In one embodiment, the tablet comprises:
a) about 269.39 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) intra-granular excipients comprising about 283.20 mg microcrystalline cellulose, about 282.50 mg mannitol, about 30 mg croscarmellose sodium, about 5 mg fumed silica, and about 10 mg sodium stearyl fumarate; and
c) the extra-granular excipient comprising 70 mg microcrystalline cellulose, about 30 mg croscarmellose sodium, and about 20 mg sodium stearyl fumarate.

In one embodiment, the tablet comprises:
a) about 150 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) intra-granular excipients comprising about 175.50 mg microcrystalline cellulose, about 175.50 mg mannitol, about 18 mg croscarmellose sodium, about 3 mg fumed silica, and about 6 mg sodium stearyl fumarate; and
c) the extra-granular excipient comprising 42 mg microcrystalline cellulose, about 18 mg croscarmellose sodium, and about 12 mg sodium stearyl fumarate.

In one embodiment, the tablet comprises:
a) about 154 mg 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt;
b) intra-granular excipients comprising about 173.47 mg microcrystalline cellulose, about 173.47 mg mannitol, about 18 mg croscarmellose sodium, about 3 mg fumed silica, and about 6 mg sodium stearyl fumarate; and
c) the extra-granular excipient comprising 42 mg microcrystalline cellulose, about 18 mg croscarmellose sodium, and about 12 mg sodium stearyl fumarate.

### ENUMERATED EMBODIMENTS

Embodiment 1: A method for treating, managing, or ameliorating a myelodysplastic syndrome comprising administering to a subject having a myelodysplastic syndrome 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate, which has the following structure:

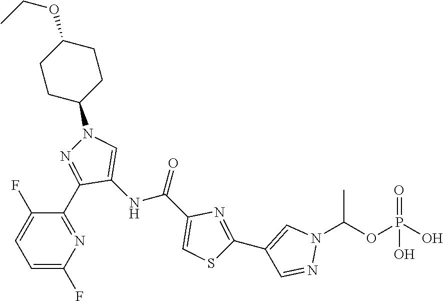 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]

or a stereoisomer or mixture of stereoisomers, a pharmaceutically acceptable salt, tautomer, solvate, hydrate, co-crystal, clathrate, or polymorph thereof (Compound 1), wherein Compound 1 is orally administered to the subject in an amount of about 100 mg to about 2000 mg per day.
Embodiment 2: The method of embodiment 1, wherein Compound 1 is an inorganic base salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.
Embodiment 3 The method of embodiment 1 or 2, wherein Compound 1 is a mono-inorganic base salt or a di-inorganic base salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.
Embodiment 4: The method of any one of embodiments 1 to 3, wherein Compound 1 is an alkali metal salt, an alkaline earth metal salt, or an ammonium salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.
Embodiment 5: The method of any one of embodiments 1 to 4, wherein Compound 1 is a mono-sodium salt, di-sodium salt, mono-potassium salt, di-potassium salt, calcium salt, magnesium salt, or mono-ammonium salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.
Embodiment 6: The method of any one of embodiments 1 to 5, wherein Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate disodium salt.
Embodiment 7: The method of any one of embodiments 1 to 5, wherein Compound 1 is 1-(4-(4-((3-(3,6-difluoropyridin-2-yl)-1-((1 r,4r)-4-ethoxycyclohexyl)-1H-pyrazol-4-yl)carbamoyl)thiazol-2-yl)-1H-pyrazol-1-yl)ethyl phosphate monosodium salt.
Embodiment 8: The method of embodiment 1, wherein Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.
Embodiment 9: The method of any one of embodiments 1 to 8, wherein the myelodysplastic syndrome is a higher-risk myelodysplastic or a lower-risk myelodysplastic syndrome.
Embodiment 10: The method of any one of embodiments 1 to 9, wherein the myelodysplastic syndrome is a higher-risk myelodysplastic syndrome.
Embodiment 11: The method of any one of embodiments 1 to 9, wherein the myelodysplastic syndrome is a lower-risk myelodysplastic syndrome.
Embodiment 12: The method of any one of embodiments 1 to 9, wherein the lower-risk myelodysplastic syndrome is refractory or relapsed lower-risk myelodysplastic syndrome.
Embodiment 13: The method of any one of embodiments 1 to 9, wherein the lower-risk myelodysplastic syndrome is lower-risk myelodysplastic syndrome that is ineligible for a prior therapy.
Embodiment 14: The method of any one of embodiments 1 to 13, wherein Compound 1 is administered once daily.
Embodiment 15: The method of any one of embodiments 1 to 13, wherein Compound 1 is administered twice daily.
Embodiment 16: The method of any one of embodiments 1 to 15, wherein the amount is about 100 mg to about 1000 mg per day.
Embodiment 17: The method of any one of embodiments 1 to 16, wherein the amount is about 100 mg per day, about 125 mg per day, about 150 mg per day, about 200 mg per day, about 250 mg per day, about 300 mg per day, about 350 mg per day, about 375 mg per day, about 400 mg per day, about 450 mg per day, about 500 mg per day, about 550 mg per day, about 600 mg per day, about 650 mg per day, about 700 mg per day, about 750 mg per day, about 800 mg per day, about 850 mg per day, about 900 mg per day, about 950 mg per day or about 1000 mg per day.
Embodiment 18: The method of any one of embodiments 1 to 16, wherein the amount is about 250 mg, about 500 mg or about 750 mg per day.
Embodiment 19: The method of any one of embodiments 1 to 18, wherein the amount is about 250 mg per day.
Embodiment 20: The method of any one of embodiments 1 to 18, wherein the amount is about 500 mg per day.
Embodiment 21: The method of any one of embodiments 1 to 18, wherein the amount is about 750 mg per day.
Embodiment 22: The method of any one of embodiments 1 to 16, wherein the amount is about 125 mg twice daily.
Embodiment 23: The method of any one of embodiments 1 to 16, wherein the amount is about 250 mg twice daily.
Embodiment 24: The method of any one of embodiments 1 to 16, wherein the amount is about 400 mg twice daily.
Embodiment 25: The method of any one of embodiments 1 to 16, wherein the amount administered is about 800 mg per day, administered in two doses, of about 400 mg each.
Embodiment 26: The method of any one of embodiments 1 to 16, wherein the amount administered is 500 mg twice daily.
Embodiment 27: The method of any one of embodiments 1 to 16, wherein the amount is about 750 mg per day administered in two doses, wherein a first dose is about 500 mg and a second dose is about 250 mg.
Embodiment 28: The method of any one of embodiments 1 to 16, wherein the amount is about 750 mg per day administered in two doses, wherein a first dose is about 250 mg and a second dose is about 500 mg.
Embodiment 29: The method of any one of embodiments 22 to 25, wherein the two doses are administered at an interval of about 6-15 hours.
Embodiment 30: The method of embodiment 26, wherein the two doses are administered at an interval of about 6, 8, 10, 12 or 15 hours.
Embodiment 31: The method of any one of embodiments 1 to 16, wherein Compound 1 is administered in an amount corresponding to about 1(0) mg per day to about 200 mg per day, about 200 mg per day to about 300 mg per day, about 450 mg per day to about 550 mg per day, or about 750 mg per day to about 850 mg per day of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.
Embodiment 32: The method of any one of embodiments 1 to 16, wherein Compound 1 is administered in an amount corresponding to about 250 mg per day, about 500 mg per day, about 750 mg per day, about 125 mg twice per day, about 250 mg twice per day, about 400 mg twice per 25 day or about 500 mg twice per day of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.
Embodiment 33: The method of any one of embodiments 1 to 32, wherein Compound 1 is administered as an oral tablet.
Embodiment 34: The method of embodiment 33, wherein the oral tablet comprises:
a) about 15% to about 35% Compound 1 by weight based on total weight of the tablet;
b) about 50% to about 70% intra-granular excipient by weight based on total weight of the tablet; and
c) about 10% to about 20% extra-granular excipient by weight based on total weight of the tablet.
Embodiment 35: The method of embodiment 33, wherein the oral tablet comprises about 100 to 300 mg of Compound 1.
Embodiment 36: The method of any one of embodiments 1 to 8, wherein the subject is relapsed, refractory/resistant, intolerant, or has an inadequate response to one or more prior therapies a myelodysplastic syndrome.
Embodiment 37: The method of embodiment 36, wherein the prior therapy comprises a treatment with an erythropoietin, luspatercept, or a hypomethylating agent.
Embodiment 38: The method of any one of embodiments 1 to 37, further comprising administering a therapeutically effective amount of a second active agent or a supportive care therapy.
Embodiment 39: The method of any one of embodiments 1 to 38, wherein the subject is 18 years or older.
Embodiment 40: The method of any one of embodiments 1 to 39, wherein the subject has failed prior lenalidomide therapy.
Embodiment 41: The method of any one of embodiments 37 to 40, wherein the hypomethylating agent is azacytidine or decitabine.
Embodiment 42: The method of any one of embodiments 1 to 41, wherein Compound 1 is administered with food.
Embodiment 43: The method of any one of embodiments 1 to 42, wherein Compound 1 is administered without food.

The following Examples are presented by way of illustration, not limitation. One skilled in the art can modify the illustrative examples to arrive at the methods.

### EXAMPLES

### Example 1

An Open-label, Phase 1b Study of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate (Compound 1C), an IRAK1/4 Inhibitor, in Patients with Lower-Risk Myelodysplastic Syndrome (LR-MDS) Who are Relapsed/Refractory/Resistant to Prior Therapies

### Rationale:

Compound 1C is an oral prodrug that is converted to the active drug N-(3-(3,6-difluoropyridin-2-yl)-1-((1r,4r)-4-ethoxycyclohexyl)-1H-pyrazol-4-yl)-2-(1H-pyrazol-4-yl)thiazole-4-carboxamide (Compound 2) in the gastrointestinal (GI) tract by alkaline phosphatase (ALP), with negligible levels of Compound 1C reaching the systemic circulation. Compound 2 is a potent and selective inhibitor of interleukin-1 receptor associated kinase 1 (IRAK1) and IRAK4 that blocks inflammatory cytokine production in response to toll-like receptors (TLR) and interleukin-1 family of receptors (IL-1R) stimulation.

It is expected that therapeutic targeting of the IRAK1/4 pathway with Compound 1C may lead to a decrease in inflammatory signaling and cell death in LR-MDS which may translate into improved hematopoiesis and result in a new potential therapeutic avenue for patients with these disorders.

### Objectives and Endpoints:

### Primary Objectives:

To assess the safety and tolerability of Compound 1C in patients with LR-MDS

To estimate the maximum tolerated dose (MTD) and/or recommended dose(s) for expansion (RDE) of Compound 1C in LR-MDS patients

### Secondary Objectives:

To assess preliminary efficacy of Compound 1C in patients with LR-MDS

To characterize the pharmacokinetics (PK) of Compound 2 and Compound 3 in patients with LR-MDS

### Exploratory Objectives:

To further assess the clinical benefit of Compound 1C in patients with LR-MDS

To assess the impact of Compound 1C on fatigue associated with anemia using a patient-reported outcome score (in dose expansion phase)

To characterize the pharmacodynamic (PD) response of interleukin-1 receptor associated kinase (IRAK) pathway related biomarkers to Compound 1C in patients with LR-MDS

### Primary Endpoints:

Incidence of adverse events (AEs)

Incidence of serious AEs (SAEs)

Incidence of selected laboratory abnormalities

Incidence of discontinuation or interruptions of Compound 1C due to AEs

Incidence of dose limiting toxicities (DLTs)

### Secondary Endpoints:

Proportion of patients with red blood cell (RBC) transfusion independence ≥8 weeks, ≥16 weeks, and ≥24 weeks

Proportion of patients with complete remission (CR) (per International Working Group [IWG] 2006)

Proportion of patients with overall response (CR, partial remission [PR], or marrow CR [mCR]) per IWG 2006

Proportion of patients with hematologic improvement (hematologic improvement [HI]-erythrocytes [E], HI-platelets [P], or HI-neutrophils [N]) per IWG 2018

Proportion of patients who achieve ≥50% reduction in number of RBC transfusions compared to baseline ≥8 weeks, ≥16 weeks and ≥24 weeks

### Duration of Response

Time to bone marrow (BM) blasts >5%

Time to progression

Time to leukemia

Change from baseline in RBC transfusions every 8 weeks

Change from baseline in platelet transfusions every 8 weeks

PK parameters of Compound 2 (active moiety of Compound 1C) and Compound 3 (3-(3,6-difluoropyridin-2-yl)-1-((1r,4r)-4-ethoxycyclohexyl)-1H-pyrazol-4-amine, inactive metabolite of Compound 2) in Cycles 1 and 2 measured by maximum plasma concentration (Cₘₐₓ), minimum plasma concentration (Cₘᵢₙ), time to maximum plasma concentration (tₘₐₓ), area under the curve (AUC) and half-life (t_{1/2}), and the metabolic ratio, calculated as AUC_{Compound 3} over AUC_{Compound 2}.

### Exploratory Endpoints

Proportion of patients with transfusion independence of both RBCs and platelets

Change from baseline using the Functional Assessment of Chronic Illness Therapy (FACIT) Fatigue Scale

Change from baseline in biomarker expression levels in plasma and BM (including, but not limited to, C-reactive protein [CRP], tumor necrosis factor [TNF]-α, interleukin [IL]-6, IL-8, macrophage inflammatory protein [MIP]1α, MIP1β, S100A8 and S100A9)

PK parameters for Compound 1C may not be generated as it is not expected to be absorbed in any significant amount.

### Overall Design:

The study is an open-label, Phase 1b study of Compound 1C to characterize safety and tolerability, to estimate the maximum tolerated dose (MTD) and/or recommended dose(s) for expansion (RDE), to characterize pharmacokinetics (PK) and to evaluate preliminary efficacy of Compound 1C in patients with LR-MDS who are relapsed, refractory/resistant, intolerant, or have had an inadequate response to prior therapies such as erythropoietins (EPOs), luspatercept, or hypomethylating agents (HMAs) for myelodysplastic syndrome (MDS).

This study is to include a Dose Escalation Phase, a Dose Expansion Phase and an exploratory Phase 1b cohort of patients with LR-MDS (see below). Up to 36 patients each in the Dose Escalation Phase and up to 36 TD LR-MDS patients in the Dose Expansion Phase are be enrolled. Dose escalation is to be performed following dose-limiting toxicity (DLT) assessments at each dose level. DLT criteria is to be used to manage toxicity during the Dose Expansion Phase. Once the RP2D is determined, an exploratory cohort of up to 10 patients with transfusion-dependent (TD) LR-MDS (ring sideroblast negative) who have not responded to or are ineligible for ESAs are to be enrolled to assess preliminary safety and efficacy in a less-heavily pretreated patient population.

All patients must complete a Screening Visit, weekly visits during the first cycle, Day 1 and Day 15 visits during subsequent 28-day treatment cycles, and an End of Study (EOS) Safety Follow-up visit (30 days±3 days after last dose). Hematologic improvement-erythroid (HI-E) responses will be assessed per IWG 2018 criteria and other responses (e.g. CR, PR) per IWG 2006 criteria, starting at week 8.

In the absence or loss of clinical benefit after ≥6 months at 250 mg QD or BID, patients may change to 500 mg QD if no prior Grade 3 or 4 toxicities requiring dose delay or reductions occurred.

### Dose Escalation Phase

The dose escalation phase is based on a modified “3+3” dose escalation scheme. The starting dose level is 250 mg once daily (QD) (Dose Level 1). Up to 6 patients are to receive Compound 1C in each dose level. The additional 3 patients in each cohort are only to be enrolled after the first 3 patients have been fully evaluated for toxicity during the DLT evaluation period of 28 days. The following criteria are to be used for the dose escalation phase:

If 0 DLTs occur in the first 3 patients, 3 patients are to be enrolled in the subsequent higher dose level. Three (3) additional patients may also be enrolled before proceeding to the next highest dose level to collect more safety and/or PK data, e.g., with agreements of the Safety Review Committee (SRC) and the Sponsor.

If 1 DLT occurs in the first 3 patients, cohort evaluation are to be postponed until 6 patients at that dose level have completed the 28 day DLT evaluation period. If 1 DLT occurs in 6 patients, dose escalation is to be continue, and 3 patients are to be enrolled in the next dose level.

If ≥2 DLTs occur in up to 6 patients enrolled at that dose level, dose escalation/cohort expansion are to be halted, and that dose level is to be considered to have exceeded the MTD.

For each subsequent higher dose level, dose escalation between cohorts is to occur after the first 3 patients in each dose level have been fully evaluated for toxicity during the DLT evaluation period of 28 days. The highest dose level with no more than 1 DLT in 6 patients are to be considered the MTD.

The table below (Table 1) shows the proposed dose levels that is to be evaluated. The dose escalation/de-escalation regimens may be adjusted per accumulating safety/efficacy/PK data, and target inhibition data from preceding cohorts. For example, the dosing schema has been revised to include BID dosing based on review of the accumulating PK and safety data.

TABLE 1

| Compound 1C | Planned Compound | Planned No. |
|---|---|---|
| Dose Level | 1C Dose | of Patients |
| Dose Level 1 | 250 mg QD | 3 to 6 |
| (starting dose) | | |
| Dose Level 2 | 500 mg QD | 3 to 6 |
| Dose Level 3 | 750 mg QD | 3 to 6 |
| Dose Level 4 | 250 mg BID | 3 to 6 |
| Dose Level 5 | 500 mg AM/250 mg PM | 3 to 6 |
| Dose Level 6 | 500 mg BID | 3 to 6 |

Abbreviations:
AM = morning dose;
BID = twice daily;
PM = evening dose;
QD = once daily;
SRC = Safety Review Committee;
TBD = to be determined

After completion of the 28-day DLT evaluation period, patients who experienced no DLTs may remain on study treatment at their respective dose level as long as they continue to demonstrate clinical benefit and/or experience no excessive toxicity. If no clinical benefits are observed up to 6 months, the patient are to discontinue study treatment, unless they are receiving 250 mg QD or BID. In the absence or loss of clinical benefit after ≥6 months at 250 mg QD or BID, patients may change to 500 mg QD if no prior Grade 3 or 4 toxicities requiring dose delay or reductions occurred.

### Dose Expansion Phase

At the completion of the Dose Escalation Phase, up to 10 additional LR-MDS patients may be enrolled in a Dose Expansion Phase to further evaluate safety and preliminary efficacy. If 2 potential doses for evaluation are chosen, then patients are to be randomized (1:1 ratio) into the 2 treatment arms (D1 and D2); otherwise, approximately 15-18 patients are to be enrolled to receive the selected RDE. The determination of the recommended dose(s) for expansion (which will not exceed the MTD), will be based on the safety, PK guidance, preliminary efficacy, and pharmacodynamic (PD) data (if available) from the Dose Escalation Phase. Stopping rules based on excessive toxicity (i.e., DLT criteria from the Dose Escalation Phase) will be used in the Dose Expansion Phase. The safety and efficacy data from the Dose Escalation and Dose Expansion Phases, along with all available clinical safety and PK/PD data, are to be used to determine the recommended Phase 2 dose (RP2D).

### Exploratory Phase 1b Cohort

Once the RP2D is determined, an exploratory cohort of up to 10 patients with TD LR-MDS (ring sideroblast negative) who have not responded to or are ineligible for ESAs are to be enrolled to assess preliminary safety and efficacy in a less-heavily pretreated patient population.

### Number of Patients:

This study is planned to include a total of up to 82 patients; up to 36 LR-MDS patients in the Dose Escalation Phase, and up to 36 LR-MDS patients in the Dose Expansion Phase, and up to 10 patients in an exploratory Phase 1b cohort.

### Intervention Groups and Duration:

The test product Compound 1C (250 mg tablets) is taken orally with or without food according to the dose levels in the table above (Table 1). In the Dose Escalation Phase, Compound 1C is to be administered in ascending dose levels with Dose level 1 initiated first. In the Dose Expansion Phase, Compound 1C is to be administered to all patients at the expansion dose(s) which will be based on PK, PD, safety, and efficacy data from the Dose Escalation Phase.

All patients must complete a Screening Visit, weekly visits during the first cycle, Day 1 and Day 15 visits during subsequent 28-day treatment cycles, and an End of Study (EOS) Safety Follow-up visit (30 days±3 days after last dose). Bone marrow collection is to be performed as described in Table 2, depending on response.

TABLE 2
Schedule of Activities

| | | | | | | | | | | | Every 3 cycles | EOS Safety |
|---|---|---|---|---|---|---|---|---|---|---|---|---|
| | | | | | | | | | | | (Cycle 6/12 weeks | Follow-up |
| | | | | | | | | | | | up to Cycle | Visit after |
| | | | | | | | | | Subsequent | | 15, and then | Discontinuation |
| | Screening¹ | Cycle 1 | | | | Cycle 2 | | Cycle 3 | Cycles | | every 6 cycles) ² | of Treatment |
| | Week/Days | | | | | | | | | | | |
| | Day −28 | | | | | | | | | | | 30 days post- |
| | to D −1 | D 1 | D 8 ² | D 15 ² | D 22 ² | D 1 ² | D 15 ² | D 1 | D 1 ² | D 15 ² | | treatment ² |
| Informed consent | X | | | | | | | | | | | |
| Medical history | X | | | | | | | | | | | |
| (including base- | | | | | | | | | | | | |
| line Hb level) ³ | | | | | | | | | | | | |
| Historical | X | | | | | | | | | | | |
| mutation status ⁴ | | | | | | | | | | | | |
| Physical | X | X* | | | | X | | | X | | | X |
| examination ⁵ | | | | | | | | | | | | |
| Height | X | | | | | | | | | | | |
| Weight | X | X* | | | | | | | | | | X |
| Vital signs ⁶ | X | X* | X | X | X | X | X | | X | X | | X |
| ECOG PS | X | | | | | | | | | | | X |
| Bone marrow and | X | | | | | | | X ⁸ | | | X ⁸ | X |
| cytogenetic | | | | | | | | | | | | |
| collection ^{7, 8} | | | | | | | | | | | | |
| Response | | | | | | | | X | | | X | X ¹⁰ |
| assessment ⁹ | | | | | | | | | | | | |
| FACIT Fatigue | | X* | | | | X | | | X | | | X |
| Scale (v4) ¹¹ | | | | | | | | | | | | |
| Hematology ^{12, 13} | X | X* | X | X | X | X | X | | X ²⁶ | X | | X |
| Blood | X | X* | X | X | X | X | X | | X ²⁶ | X | | X |
| chemistry ^{13, 14} | | | | | | | | | | | | |
| Hepatitis | X ²⁷ | | | | | | | | | | | |
| screening tests | | | | | | | | | | | | |
| Urinalysis ^{13, 15} | X | X* | | | | X | | | | | | X |
| Coagulation | X | X* | | | | X | | | | | | X |
| tests ^{13,} ¹⁶ | | | | | | | | | | | | |
| Pregnancy test ¹⁷ | | X* | | | | | | | | | | X |
| 12-lead ECG ¹⁸ | X | X* | X | | | X | | | X | | | X |
| Blood samples for | | X ^{20, 21} | X ²¹ | | | X ^{19, 21} | | | X | | | |
| PK ¹⁹ | | | | | | | | | | | | |
| Biomarkers ²² | | X* | | | | X | | | X | | | |

| | | |
|---|---|---|
| Study events | X | Compound 1C administration ²³ |
| Adverse events | X | X |
| Prior and | X | X |
| concomitant | | |
| medications | | |
| Prior and | X ²⁴ | X |
| concomitant | | |
| blood transfusions | | |
| Prior LR- | X | |
| MDS | | |
| treatments ²⁵ | | |

Abbreviations: BM = bone marrow; BP = blood pressure; CRP = C-reactive protein; ECG = electrocardiogram; ECOG = Eastern Cooperative Oncology Group; EOS = end of study; ICF = informed consent form; INR = international normalized ratio; IWG = International Working Group; MIP = macrophage inflammatory protein; PCR = polymerase chain reaction; PD = pharmacodynamic; PK = pharmacokinetic; PS = performance status; PT = prothrombin, PTT = partial thromboplastin time; TNF = tumor necrosis factor.
*Indicates pre-dose evaluation. All results (except Biomarkers and FACIT-Fatigue scale results) must be reviewed prior to C1D1 dosing.
¹Screening includes retrospective collection of baseline hemoglobin and transfusion information which can be collected after the patient signs ICF.
² Visits with a window of ±3 days.
³ Baseline Hb up to 16 weeks prior to Day 1.
⁴ The patient's most recent available mutation status may be collected from historical medical records for baseline disease characterization.
⁵ A complete physical examination with height measurement will be conducted at Screening. An abbreviated or directed physical examination will be conducted on Day 1 of each subsequent cycle at the clinic visit, and at the EOS visit. For Cycle 1 Day 1, physical examination may be performed within a window of −3 days to facilitate the scheduling of PK tests.
⁶ BP (systolic and diastolic), heart rate, oral temperature, and respiratory rate. If it is not feasible for patients to obtain vital sign measurements at their clinical site, vital signs may be obtained by the patient's local clinician for the following study visits: Dose escalation phase - Day 15 visits at Cycle 2 and Subsequent Cycles; Dose expansion phase - Day 15 and Day 22 visits at Cycle 1, Cycle 2, and Subsequent Cycles.
⁷ Historical bone marrow aspirate and/or biopsy collected within 1 month prior to the first dose of study drug do not need to be repeated.
⁸ Bone marrow aspirate and/or biopsy on Day 1 of Cycle 3 of therapy and every 8 weeks until transfusion independence is achieved. In patients who have achieved transfusion independence, BM biopsies will be performed approximately every 3 months during the first 6 months of therapy and then, if persistent response, every 6 to 12 months (or as clinically indicated). Cytogenetics should be collected any time a bone marrow exam is performed.
⁹ MDS disease assessment should be completed by the investigator in conjunction with the bone marrow aspirate/biopsy and cytogenetics. Response assessments will be performed following IWG 2006 criteria and IWG 2018 criteria.
¹⁰ EOS bone marrow aspiration/biopsy should only be performed if it's been at least 4 months since the previous one.
¹¹ FACIT-Fatigue assessments will be performed at baseline (C1D1) and D 1 of every subsequent cycle for the expansion phase of the study only.
¹² Hematology includes weekly (Cycle 1) and bi-weekly (subsequent cycles) red blood cells, white blood cells, platelets, neutrophils, lymphocytes, monocytes, basophils, eosinophils, hemoglobin, and hematocrit and reticulocyte count.
¹³ If it is not feasible for patients to obtain safety laboratory measurements at their clinical site, safety labs may be obtained by the patient's local laboratory for the following study visits: Dose escalation and expansion phase - Day 15 visits at Cycle 2 and Subsequent Cycles.
¹⁴ Chemistry includes sodium, potassium, chloride, bicarbonate, calcium, phosphate, blood urea nitrogen (BUN), creatinine, glucose, lactate dehydrogenase (LDH), aspartate aminotransferase (AST), alanine aminotransferase (ALT), alkaline phosphatase, total (indirect and direct) bilirubin, total protein, albumin.
¹⁵ Urinalysis includes specific gravity pH, glucose, protein, blood, ketones, by dipstick. Microscopic examination (if blood or protein is abnormal).
¹⁶ Coagulation tests include PT, PTT, and INR.
¹⁷ Serum Pregnancy testing will be conducted for women of childbearing potential at Screening and urine tests will be conducted at all other visits.
¹⁸ 12-lead ECGs to be collected at screening, pre-dose and post-dose (+3 hr ± 30 minutes) on Day 1 Cycle 1, post-dose (+3 hr ± 30 minutes) on Day 8 of Cycle 1, Day 1 of Cycle 2, Day 1 of each subsequent cycle, and at the EOS visit.
¹⁹ Serial plasma samples for PK analysis will be collected from all patients during Dose Escalation Phase and Dose Expansion Phase up to Cycle 6 only.
²⁰ The PK samples collected on Cycle 2, Day 1 are intended for steady state PK analysis. In the event of a dosing interruption during the Cycle 1 dosing period, the patient's Cycle 2, Day I samples should be collected about 28 days after re-initiating study drug.
²¹ Serial plasma samples for PK analysis will be collected from all patients during the dosing period on Day 1 and Day 8 of Cycle 1 and on Day 1 of Cycle 2 for assessment of Compound 1C, Compound 2, and Compound 3 concentrations (Dose Escalation and Dose Expansion Phase).
²² Plasma samples for exploratory biomarker assessment (CRP, TNF-α, IL-6, IL-8, MIP1α, MIP1β, S100A8, S100A9, and possibly others) to be collected from all patients during Dose Escalation Phase and Dose Expansion Phase up to Cycle 6 only.
²³ Compound 1C (250 mg tablets) is taken orally with or without food according to the dose levels in Table 1. In the Dose Escalation Phase, Compound 1C will be administered in ascending dose levels with Dose level 1 initiated first. In the Dose Expansion Phase, Compound 1C will be administered to all patients at the expansion dose(s) which will be based on PK, PD, safety, and efficacy data from the Dose Escalation Phase. Patients may remain on study treatment as long as they continue to demonstrate clinical benefit and no excessive toxicity.
²⁴ Prior blood transfusions will be collected for up to 16 weeks prior to Day 1.
²⁵ Prior LR-MDS treatments includes collection of all prior lines of LR-MDS treatment.
²⁶ Intra-patient dose escalation patients will need to have safety laboratory tests done on D 1, D 8, and D 15 of the initial cycle at the higher dose.
²⁷ Includes hepatitis B surface antigen (HBsAg), hepatitis B core antibody, and hepatitis C virus (HCV) antibody with reflex to quantitative real-time PCR (qPCR).

Patients may remain on study treatment at their respective dose level as long as they continue to demonstrate clinical benefit and experience no excessive toxicity (e.g., no clinically significant study treatment-related Grade >3 toxicity).

Patients who have not shown any clinical benefits—e.g., not achieved durable transfusion (RBC or platelet) independence (i.e., ≥8 weeks), reduced transfusion requirements from baseline, or achieved any hematologic improvement HI) per International Working Group (IWG) 2018 criteria (Platzbecker 2019) by Month 6 must discontinue study treatment, unless they are receiving 250 mg QD or BID.

In the absence or loss of clinical benefit after ≥6 months at 250 mg QD or BID, patients may change to 500 mg QD if no prior grade 3 or 4 toxicities requiring dose delay or reductions occurred.

### Study Population

Prospective approval of protocol deviations to recruitment and enrollment criteria, also known as protocol waivers or exemptions, is not permitted.

### Inclusion Criteria

Patients are eligible to be included in the study only if all of the following criteria apply:
1. Must be ≥18 years of age at the time of signing the informed consent.
2. Must have definitive diagnosis of MDS with very low, low, or intermediate-1 risk (International Prognostic Scoring System [IPSS]-R≤3.5) and ≤5% BM myeloblasts.
3. Must be relapsed, refractory/resistant, intolerant, or have inadequate response to therapies with known clinical benefits for MDS, such as, EPOs, luspatercept, and HMAs (i.e., azacytidine or decitabine). Patients with del (5q) must have failed prior lenalidomide therapy.
4. DOSE ESCALATION PHASE:
a. Must meet at least one of the following criteria prior to initial administration of study treatment:
i. Symptomatic anemia with hemoglobin ≤9.0 g/dL and no RBC transfusion within 16 weeks of registration, or
ii. RBC transfusion dependent defined as receiving ≥2 units of packed red blood cells (PRBCs) within 8 weeks in the preceding 16 weeks for a hemoglobin <9.0 g/dL
5. DOSE EXPANSION PHASE.
a. Relapsed, refractory to or ineligible for ESAs and has previously received one or more approved therapies for LR-MDS
b. Must be RBC transfusion dependent defined as receiving ≥2 units of packed red blood cells (PRBCs) within 8 weeks in the preceding 16 weeks for a hemoglobin <9.0 g/dL
6. EXPLORATORY PHASE 1b COHORT:
a. Transfusion-dependent LR-MDS (ring sideroblast negative) who have not responded to or are ineligible for ESAs.
7. All patients must have documented marrow iron stores. If marrow iron stain is not available, the transferrin saturation must be >20% or a serum ferritin >100 ng/mL.
8. Must have Eastern Cooperative Oncology Group (ECOG) performance status 0 to 2 at screening.
9. Must have adequate organ function, defined as:
a. Hepatic function:
i. AST and ALT ≤1.5≤upper limit of normal (ULN)
ii. total bilirubin ≤1.5≤ULN
b. Renal function defined as creatinine clearance >60 mL/min (using Cockcroft-Gault), or blood creatinine <1.5 mg/dL
10. Female patients must be either postmenopausal for at least 1 year or surgically sterile; or, if of childbearing potential, must not be pregnant or lactating and must agree to use 2 highly effective methods of birth control throughout the duration of the trial and for 30 days following the last dose.
11. Male patients must be willing to use adequate contraception, and to refrain from sperm donation, from the time of dosing until 90 days after the last dose of study treatment.
12. Willing and able to provide signed informed consent which includes compliance with the requirements and restrictions listed in the informed consent form (ICF) and in this protocol.

### Exclusion Criteria

Patients are excluded from the study if any of the following criteria apply:
1. Prior treatment for MDS (i.e., TPOs, EPOs, luspatercept, HMAs) concluded <4 weeks prior to study treatment.
2. Clinically significant anemia resulting from iron, B12 or folate deficiencies, autoimmune or hereditary hemolysis, or GI bleeding.
3. MDS secondary to treatment with radiotherapy, chemotherapy, and/or immunotherapy for malignant or autoimmune diseases.
4. Diagnosis of chronic myelomonocytic leukemia.
5. History of uncontrolled seizures.
6. Uncontrolled bacterial or viral infection (i.e., documented HIV, hepatitis B or hepatitis C).
7. History of an active malignancy within the past 2 years prior to study entry, with the exception of:
a. Adequately treated in situ carcinoma of the cervix uteri
b. Adequately treated basal cell carcinoma or localized squamous cell carcinoma of the skin, or
c. Any other malignancy with a life expectancy of more than 2 years
8. History of or active, clinically significant, cardiovascular, respiratory, GI, renal, hepatic, neurological, psychiatric, musculoskeletal, genitourinary, dermatological, or other disorder that, in the Investigator's opinion, could affect the conduct of the study or the absorption, metabolism or excretion of the study treatment.
9. Prior history of autologous or allogeneic stem cell transplantation.
10. Marked baseline prolongation of QT/QTc interval (e.g., repeated demonstration of a QTc interval >480 milliseconds [msec]) (Common Terminology Criteria for Adverse Events [CTCAE] Grade 1) using Fridericia's QT correction formula.
11. History of additional risk factors for TdP (e.g., symptomatic heart failure with left ventricular ejection fraction [LVEF] ≤40%, hypokalemia, family history of Long QT Syndrome).
12. Receiving any other concurrent chemotherapy, radiotherapy, or immunotherapy within 2 weeks of initiating study treatment, or the toxicity of the relevant prior treatment has not been resolved yet.
13. Use of concomitant medications that prolong the QT/QTc interval during study treatment.
14. Use of concomitant medications that are strong CYP3A or CYP2B6 inhibitors or inducers during study treatment.
15. Receipt of any other investigational agent or treatment within 2 weeks of initiating study treatment, or the toxicity of the relevant prior treatment has not been resolved yet.
16. Unable or unwilling to cooperate with the Investigator for any reason.

### Pharmacokinetic Rationale

The starting dose (250 mg QD) was proposed based on simulations, using a population PK model of Compound 2 after Compound 1C administration in healthy volunteers and a PK/PD model relating Compound 2 exposure to cytokine inhibition after a LPS challenge study in healthy volunteers. The 250 mg QD Compound 1C dosing was predicted to result in daily Compound 2 exposure levels of approximately 4,545 ng*h/mL (well below the daily AUC of 8,490 ng*h/mL, as defined in the 13-week repeated-dose toxicity study in rats, estimated at the 5 mg/kg/day NOAEL dose level). At this predicted AUC level, a ≥25% release inhibition of cytokines is expected to last for over 8 hours during the 24-hour dosing period.

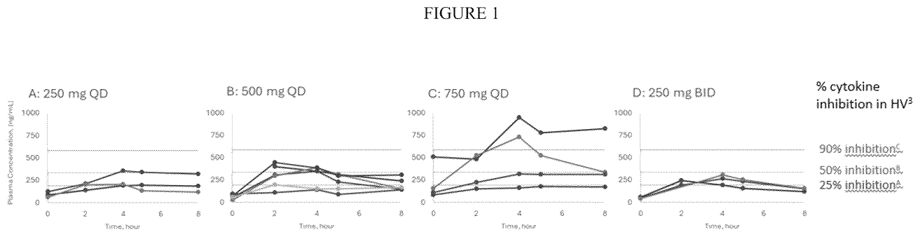

As can be seen in FIG. 1, panels A-D. Compound 2 concentrations in LR-MDS patients receiving 250 mg QD are below the concentrations presumed to have a minimal inhibiting effect on LPS-induced cytokine release. At the higher doses of 500 mg QD and 750 mg QD, Compound 2 plasma concentrations in most LR-MDS patients reach levels at which 50% and 90% LPS-induced cytokine inhibition may be achieved in HV (FIG. 1, panel B and panel C).

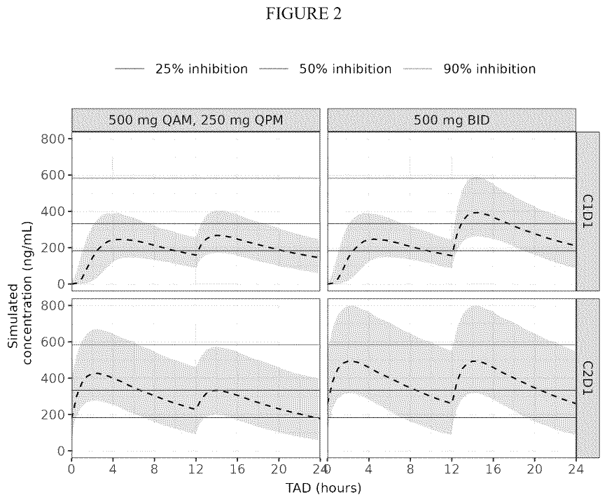

As Compound 2 concentrations were subsequently found to be lower in LR-MDS patients than predicted by HV, a new PK model using LR-MDS patient data has been developed to predict Compound 2 concentrations and exposures for the 500/250 mg split dose (dose level 5) and 500 mg BID (new dose level 6). Expected Compound 2 concentration profiles for these two dose regimens are shown in FIG. 2, while the predicted Compound 2 exposure levels are shown in Table 3.

TABLE 3
Predicted Noncompartmental PK Parameters
of 2 Planned Dosing Regiments

| Regimen | AUC_{0-24, ss} (h*ng/mL) | C_{max, ss} (ng/mL) |
|---|---|---|
| 500 QAM, 250 QPM | 7,105 | 427 |
| 500 mg BID | 9,231 | 494 |

Abbreviations: AUC_{0-24, ss}: Area under the curve from time zero to 24 hours at steady state; BID = twice daily, C_{max, ss} = maximum concentration at steady state; QAM = single dose in the morning; QPM = single dose in the evening.

Based on the simulation results, dose level 5 (500 mg/250 mg split dose) and dose level 6 (500 mg BID) are expected to provide concentrations of Compound 2 that may result in clinically meaningful cytokine inhibition, and potentially better clinical outcomes in LR-MDS patients

### Summary of Preliminary Safety and Efficacy Data

The dose escalation cohorts have previously been revised to include “twice-daily” dosing (dose level 4, 250 mg BID and dose level 5, 500 mg/250 mg split dose) based on review of the cumulative PK and safety data, suggesting that a BID dosing regimen may provide more sustained exposures of the active drug Compound 2 that may enhance efficacy.

As of the first data cutoff date, 20 patients were enrolled in the first 4 dose levels: 250 mg QD (n=3), 500 mg QD (n=6), 750 mg QD (n=6), and 250 mg BID (n=5). The median age was 76 (range 50-83): 13 (65%) were males. The median number of prior therapies was 4 (range 1-8). Prior therapies included luspatercept [n=16 (80%)] and an HMA [n=15 (75%)]. The majority of patients were high transfusion burden (≥8u RBCs/prior 16 weeks) at study entry (n=15; 75%); 3 patients (15%) were low transfusion burden (3-7u RBCs/prior 16 weeks) and 2 (10%) were non-transfused.

In general, Compound 1C was well-tolerated. The most frequent (?15%) treatment-related adverse events (TEAEs) were diarrhea and fatigue (both n=5; 25%), nausea (n=4; 20%), and anemia, constipation, cough, dyspnoea, pneumonia, pruritus and vomiting (n=3 each; 15%); all were grade (G)1/2 except for anemia and pneumonia. The most frequent G3/4 adverse events (AEs) were pneumonia, anemia. ALT increase and upper GI hemorrhage (all n=2; 10%). No G3/4 AEs were reported in the 250 mg QD dose group, 1 was reported in the 250 mg BID dose group, and the incidence of G3/4 AEs was generally balanced between the 500 and 750 mg QD dose groups, with the exception of 2 reports of G3/4 transaminase (ALT and AST) elevations in the 750 mg QD dose group. AEs leading to treatment discontinuation were acute cardiac failure (500 mg QD dose group) and AST/ALT increased (750 mg QD dose group). The most common AEs leading to dose modification were pneumonia (n=3) and ALT/AST increases (n=2).

At the first data cutoff, 14 patients were evaluable for response (i.e. received ≥1 dose of study drug with ≥1 efficacy assessment). Among the 6 non-evaluable patients, 1 patient withdrew due to an AE and 5 patients had <8 weeks follow-up (i.e., too early to evaluate for response). No responses occurred at 250 mg daily. Three patients achieved RBC-transfusion independence (RBC-TI) for ≥8 weeks: 1 receiving 500 mg QD and 2 receiving 750 mg QD. The median duration of RBC-TI was 28.9 weeks (range 12.4-39.9 weeks). Durable RBC-TI >24 weeks was achieved in 2 HTB patients for 28.9 and 39.9 weeks respectively). One additional patient achieved a minor HI-E response (≥50% reduction in RBC units in 16 weeks). At doses 500 mg QD, Compound 2 plasma concentrations at steady state reached or exceeded concentrations correlating with 50% (n=6) or 90% (n=3) LPS-induced cytokine inhibition as observed in healthy volunteers.

Taken together, the PK and safety data support the addition of a sixth dose level, 500 mg BID, to further characterize the safety and PK of Compound 1C in LR-MDS patients and optimize the selection of the recommended dose(s) for further expansion. Although daily Compound 2 exposure after the 750 mg QD dose and estimated daily AUC for the 250 mg BID are similar at steady state (2,880 ng*h/mL after the 750 mg QD dose vs. 2×1,460=2,920 ng*h/mL after the 250 mg BID dose, respectively), peak exposure may be more relevant for activity. Accordingly, it is proposed that patients receiving 250 mg daily or 250 mg BID that are not showing signs of clinical benefit after 6 months be allowed to switch to a 500 mg daily dose.

As of the second data cutoff date, 22 patients were enrolled in the first 5 dose levels: 250 mg QD (n=3), 500 mg QD (n=6), 750 mg QD (n=6), 250 mg BID (n=6) and 500/250 mg QD (n=1). The median age was ≥75 years (range 50-84); 14 were male and 8 were female. The majority of patients were low risk MDS at study entry (n=164; 64%); and 8 patients (36%) were intermediate risk MDS. The median number of prior therapies was 3 (range 1-8). Prior therapies included luspatercept [n=17 (77%)] and an HMA [n=16 (73%)]. The majority of patients were high transfusion burden (≥8 units RBCs/prior 16 weeks) at study entry (n=16; 73%); 4 patients (18%) were low transfusion burden (3-7 units RBCs/prior 16 weeks) and 2 (9%) were non-transfused. Mean baseline absolute neutrophil count (ANC) was 2.2×10⁹/L (range 0.3-5.4×10⁹/L). Mean baseline platelet count was 168×10⁹/L (range 31.5-509.5×10⁹/L).

Treatment-related adverse events occurring in ≥2 patients by dose level are provided below in Table 4:

TABLE 4

| | 250 mg QD | | 500 mg QD | | 750 mg QD | | 250 mg BID | | 500/250 mg QD | | Total | | |
|---|---|---|---|---|---|---|---|---|---|---|---|---|---|
| Preferred | (n = 3) | | (n = 6) | | (n = 6) | | (n = 6) | | (n = 1) | | (n= 22) | | |
| term | All | G3/4 | All | G3/4 | All | G3/4 | All | G3/4 | All | G3/4 | All | | G3/4 |
| Diarrhea | 0 | 0 | 1 (17%) | 0 | 2 (33%) | 0 | 0 | 0 | 0 | 0 | 3 | (14%) | 0 |
| Dyspnoca | 0 | 0 | 1 (17%) | 0 | 2 (33%) | 0 | 0 | 0 | 0 | 0 | 3 | (14%) | 0 |
| Nausea | 0 | 0 | 0 | 0 | 3 (50%) | 0 | 0 | 0 | 0 | 0 | 3 | (14%) | 0 |
| Neut. count | 0 | 0 | 0 | 0 | 2 (33%) | 1 (17%) | 0 | 0 | 1 | 0 | 3 | (14%) | 1 (4%) |
| decreased | | | | | | | | | | | | | |
| ALT incr.* | 0 | 0 | 0 | 0 | 2 (33%) | 2 (33%) | 0 | 0 | 0 | 0 | 2 | (9%) | 2 (9%) |
| AST incr.* | 1 (33%) | 0 | 0 | 0 | 2 (33%) | 1 (17%) | 0 | 0 | 0 | 0 | 2 | (9%) | 1 (4%) |
| Constipation | 0 | 0 | 0 | 0 | 2 (33%) | 0 | 0 | 0 | 0 | 0 | 2 | (9%) | 0 |

*Dose limiting toxicity: G3/4 ALT/AST increase in 1 patient at 750 mg QD.
AE, adverse event; ALT, alanine aminotransferase; AST, aspartate aminotransferase; BID, twice daily; G, grade; incr, increased; neut, neutrophil; QD, once daily.

Summary of responses is provided below in Table 5:

TABLE 5

| Pt | Sex, | Dose | BL | | HI-E | Response |
|---|---|---|---|---|---|---|
| ID | age | level | RBCS | Prior therapies | response | duration |
| 4 | 76, M | 500 mg | HTB | ESA, canakinumab, | TI | 51.9 w |
| | | QD | | ALK2 inhibitor, | | |
| | | | | decitabine | | |
| 5 | 75, M | 500 mg | HTB | ESA, azacitidine, | 64% ↓ in | |
| | | QD | | luspatercept, | pRBCs | |
| | | | | fostamatinib, | | |
| | | | | anti-TIM-3 ab | | |
| 10 | 59, M | 750 mg | HTB | Azacitidine, | TI | 28.9 w |
| | | QD | | lenalidomide, | | |
| | | | | luspatercept | | |
| 11 | 50, F | 750 mg | LTB | Darbepoetin, | TI (+marrow | 8.1 w |
| | | QD | | luspatercept | CR) | |

Ab, antibody; ALK, anaplastic lymphoma kinase; BID, twice daily; CR, complete response; ESA, erythropoiesis-stimulating agent; F, female; HTB, high transfusion burden; ID, identification; LTB, low transfusion burden; M, male; pRBCs, packed red blood cells; Pt, patient; QD, once daily; RBC, red blood cell; TI, transfusion independence; TIM-3, T-cell immunoglobulin and mucin domain-containing protein 3; w, weeks.

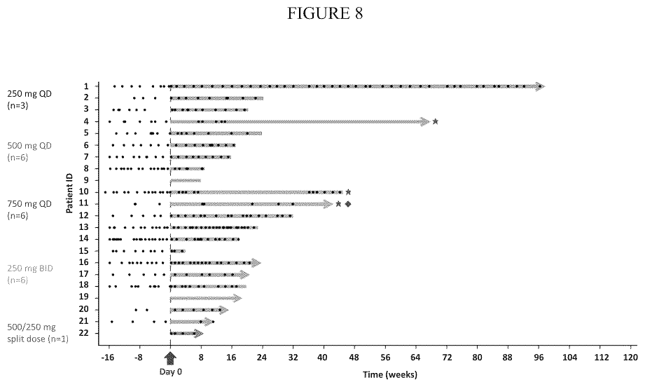

FIG. 8 provides red blood cell transfusion frequency by dose level of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

In certain embodiments, Compound 1 was well-tolerated in the older patient population. In certain embodiment, the patients were high transfusion burden (HTB, 38 units RBCs) with failed prior therapies. In certain embodiments, the incidence of G3/4 cytopenias and infections in the treated patients was low.

In one embodiment, 36% patients receiving Compound 1 doses ≥500 mg QD achieved RBC-transfusion independence (RBC-TI)/Hematologic improvement-erythroid (HI-E) responses. In one embodiment, durable RBC-transfusion independence (>6 months) was achieved.

In certain embodiments, Compound 1 was well-tolerated in the heavily pretreated LR-MDS patient population. In certain embodiments, the patients were high transfusion burden (HTB, ≥8 u RBCs). In certain embodiments, the incidence of G3/4 cytopenias and infections in the treated was low.

In certain embodiment, hematologic responses, red blood cell-transfusion independence (RBC-TI)/hematologic improvement-erythroid (HI-E) were observed in 40% transfusion dependent patients receiving Compound 1C doses ≥500 mg QD, with durable responses (>24 weeks). In certain embodiments, the patients had received prior treatment with hypomethylating agent (HMA).

### Example 2: a Nonlinear Mixed-Effect Pharmacokinetic Model of Compound 2, a Novel Dual IRAK1/4, in Healthy Volunteers after Administration of the Prodrug Compound 1C

### Study Design

In this study, Compound 1 was administered in a capsule formulation as single (Part I: 20, 80, 250, 500, and 960 mg; n=25) or multiple doses (Part II: 500 and 960 mg BID, 14 days, n=12) to healthy volunteers. In Part III of the study, an additional four subjects received single doses of 750 mg in capsules (fasting), and 750 mg in a tablet formulation (fasting and fed), with one week wash-out periods between the three doses.

### Data

A total of 996 samples were collected from 41 subjects receiving Compound 1 capsules in Parts I, II, and ill. Using validated bioanalytical methods, samples were analyzed for Compound 1, Compound 2, and Compound 3 content. Compound 1 was measurable in a handful of samples only, with all concentrations close to the limit of quantification (LOQ); Therefore, no PK analysis was performed on Compound 1 data. Compound 2 and its metabolite Compound 3 were quantifiable in 931 and 835 samples, respectively.

Compound 2 and Compound 3 plasma concentration were used to develop a compartmental PK model utilizing a nonlinear mixed-effect modeling approach. First, separate models were developed for each analyte. The models were subsequently combined, using a fixed Compound 2/Compound 3 conversion factor. A subset of data not used in model development, at a dose level not included in the original data set (single 750 mg dose of Compound 1, administered in a capsule formulation), was reserved for model validation.

### Methods and Results

Individual concentration-time data were used to generate various graphs and analyzed using a noncompartmental analysis (NCA) approach and the NCA parameters were evaluated to guide the development of the compartmental model.

### Graphical Evaluation and NCA

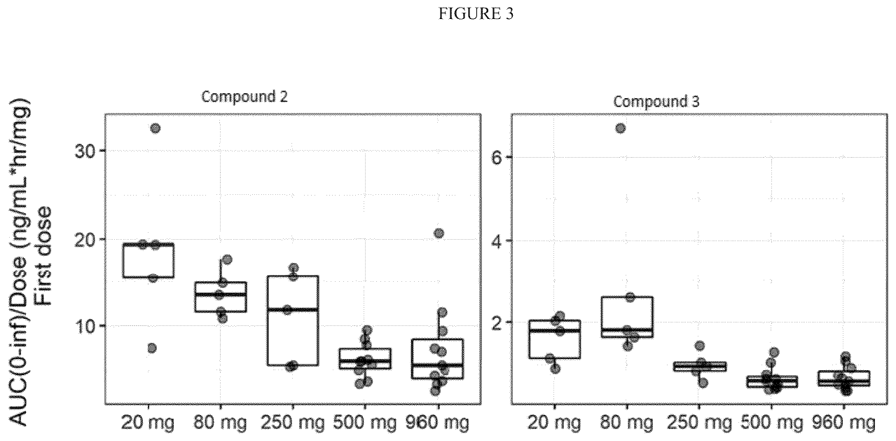

Results of the NCA indicate less-than-proportional increases in Compound 2 and Compound 3 Cmax and AUCs with increasing Compound 1C doses. FIG. 3 shows dose-normalized Cₘₐₓ and AUC_{0-inf} estimates after a single dose, including the first dose of the MAD cohorts, for both Compound 2 and Compound 3. There were no differences in elimination half-life estimates for each analyte (approximately 15 hours for Compound 2 and 20 hours for Compound 3) across the tested dose levels, implying no differences in disposition or elimination of the two analytes across different dose levels. Thus, a decrease in bioavailability of Compound 2 with dose was assumed to be the reason for the observed nonlinearity in exposure for both Compound 2 as well as its metabolite, Compound 3.

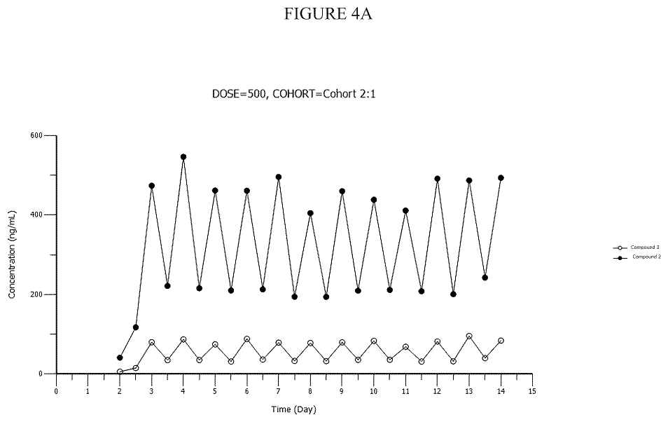

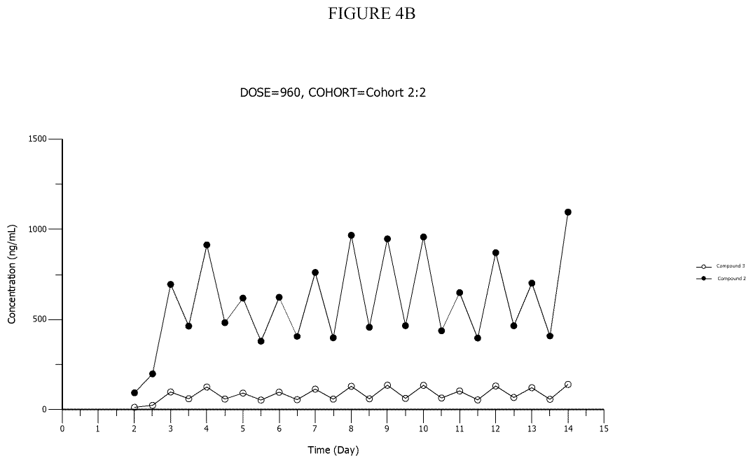

Morning trough concentrations were higher than the evening levels for both Compound 2 and Compound 3 in all collected samples after BID dosing (FIGS. 4A and 4B). It is to be noted that the morning samples were collected after the dose on the prior evening, administered 2 hours after dinner, while the evening samples were collected after the morning dose, administered after a minimum of 10 hours fasting.

All individual Compound 2 and Compound 3 data from Parts I and II of the study were used in the model building exercise, using the first order method with interaction (FOCE) in NONMEM, version 7.4.3 (Icon Development Solutions, United States). The influence of BQL observations were captured by utilizing the Laplacian option to execute the M3 likelihood based method. Changes in Akaike information criterion (AIC), visual inspection of diagnostic scatter plots, condition number, successful convergence of the minimization routine with at least 2 significant digits, and the precision and plausibility of parameter estimates guided model selection. Compound 2 and Compound 3 data following capsule administration in Part III of the study were used as an external validation of the model, using virtual predictive checks (VPC).

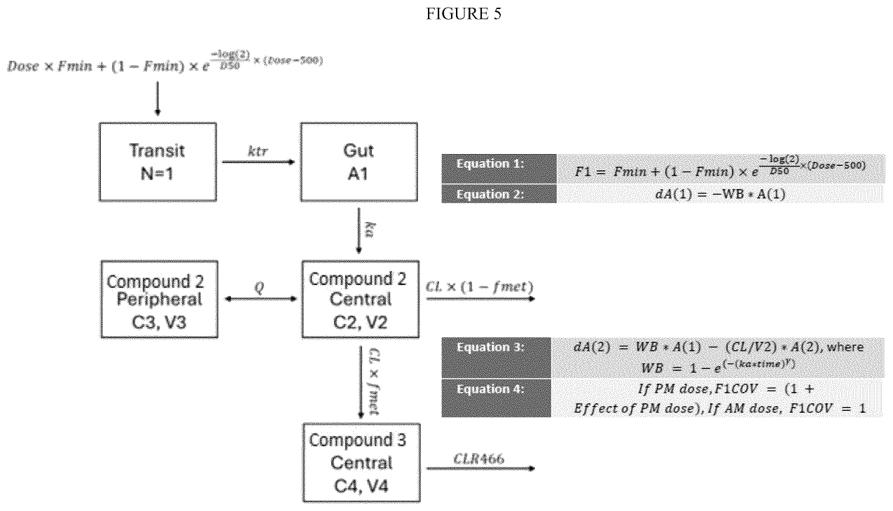

The final model consisted of a two-compartment model describing Compound 2 concentration-time data, with two elimination pathways, one for transformation to Compound 3 and one accounting for other elimination (FIG. 5). The fraction of metabolism to Compound 3 was fixed to the ratio of Compound 3 AUC over Compound 2 AUC, equal to 0.125. Compound 3 pharmacokinetics was best described by a one compartment model. A nonlinear system, normalized to the 500 mg dose, was used to describe the decrease in Compound 2 bioavailability with increasing Compound 1C dose (eq. 1). Compound 2 absorption was described by a Weibull-like model (eq. 2,3), including a transient compartment. Furthermore, different bioavailability parameters were assumed for morning and evening doses to account for the higher Compound 2 morning trough concentrations (eq. 4). Model parameter estimates are presented in Table 6.

TABLE 6

| | | | Estimate | | BSV, % CV |
|---|---|---|---|---|---|
| Model | Parameter | Description | (% RSE) | | (% Shrinkage) |
| Weibull | ka | Absorption rate (1/h) | 0.882 | (12.5%) | 63.4 (4) |
| Absorption | Υ | Shape parameter | 2.62 | (8.1%) | — |
| Compound 2 | CL | Clearance of | 177 | (8.1%) | 36.2 (2) |
| Disposition | Compound 2 | Compound 2 | | | |
| | (L/h) | | | | |
| | V2 (L) | Central volume of | 1580 | (12%) | 57.4 (7) |
| | | distribution | | | |
| | Q (L/h) | Intercompartmental | 615 | (19.2%) | — |
| | | clearance | | | |
| | V3 (L) | Peripheral volume of | 1840 | (11.2%) | — |
| | | distribution | | | |
| | Fmet | Fraction of Compound 2 | 0.125 FIXED | | 33.2 (3) |
| | | metabolized to | | | |
| | | Compound 3 | | | |
| | MTT (h) | Mean transit time | 0.02 | (11.1%) | — |
| Compound 3 | CL | Compound 3 Clearance | 184 | (7.2%) | — |
| Disposition | Compound 3 | | | | |
| | (L/h) | | | | |
| | V4 | Central Volume, | 19.2 | (37.3%) | — |
| | | Compound 3 | | | |
| Weibull | Fmin | Maximum decrease in | 0.985 | (0.4%) | — |
| Absorption | | relative F | | | |
| | D50 | Dose causing 50% | 69.6 | (6.9%) | — |
| | | reduction in F | | | |
| | F1COV | If PM dose, F1COV = | 3.23 | (9.7%) | — |
| | | (1 + Effect of PM dose) | | | |
| | | If AM dose, F1COV = 1 | | | |
| Residual | CV | Proportional Error (CV) | 29.8% | (5.1%) | — |
| Error | Compound 2 | | | | |
| | SD | Additive Error (ng/mL) | 0.576 | (23.3%) | — |
| | Compound 2 | | | | |
| | CV | Proportional Error (CV) | 29.2% | (4.7%) | — |
| | Compound 3 | | | | |
| | SD | Additive Error (ng/mL) | 0.381 | (22.8%) | — |

Compound 3

In certain embodiments, the PK properties of Compound 2 and its metabolite Compound 3 in healthy volunteers receiving various single and multiple doses of the prodrug Compound 1C can be described based on the integrated nonlinear mixed-effect model provided herein. In certain embodiments, the model includes features, accounting for changes in Compound 2 bioavailability with dose or food intake.

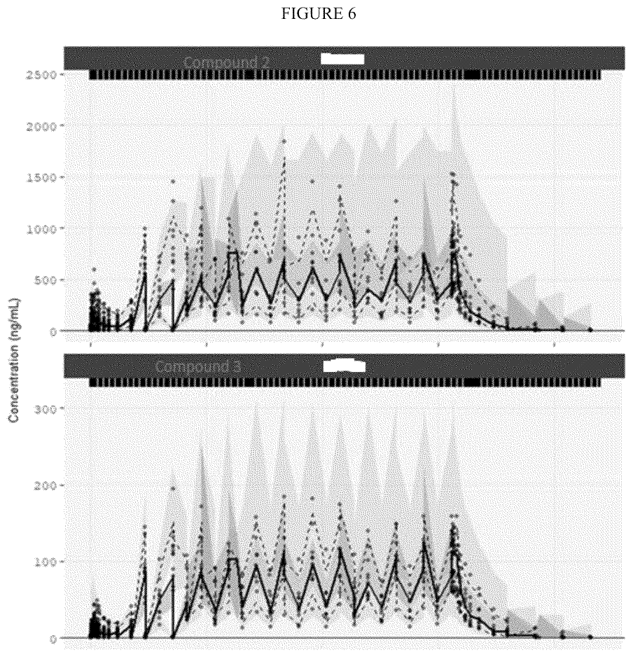

In certain embodiments, the model provided herein describes the data well, accounting for the nonlinear bioavailability and the morning/evening trough concentration differences, as evident by the visual predictive check plots (FIG. 6).

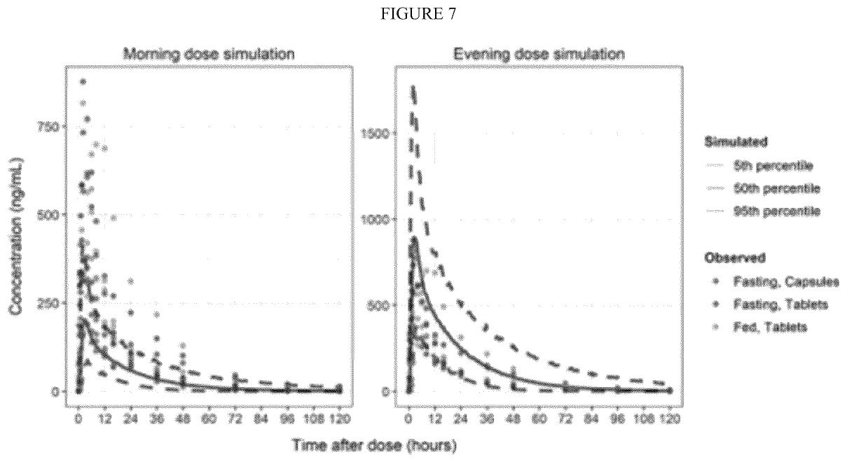

In certain embodiments, the model provided herein could predict Compound 2 and Compound 3 concentrations after a single Compound 1C dose with neither the dose level (750 mg) nor the concentration data included in the original data set used during the model development process (FIG. 7).

### Example 3: a Phase 1, Single-Center, Randomized, Crossover Study to Investigate the Effect of Food on Compound 2 Pharmacokinetics after a Single Doses of Compound 1C in Healthy Subjects

### Objectives and Endpoints:

### Primary Objectives:

To investigate the effect of high fat meal on the pharmacokinetic exposure properties of Compound 2 (Cₘₐₓ and AUC_{0-inf}) after a single oral dose of Compound 1C in a healthy subject.

### Secondary Objectives:

To assess the safety and tolerability of Compound 1C after single oral administration

To characterize the pharmacokinetics of Compound 1C (prodrug), Compound 2 (active moiety), and Compound 3 (3-(3,6-difluoropyridin-2-yl)-1-((1r,4r)-4-ethoxycyclohexyl)-1H-pyrazol-4-amine, inactive metabolite of Compound 2).

### Design:

This is a randomized, 2×2 crossover study with two-sequence and two-period. A single dose of 500 mg Compound 1C will be administered without (fasting, reference) or with food (fed), with a minimum of one week washout period between the two administrations. A total of 12 evaluable subjects are planned with 6 each assigned to 1 of the 2 arms.

TABLE 7

Study Day

| Sequence | Day 1 | Day 2-7 | Day 8 |
|---|---|---|---|
| Sequence 1 N = 6 | Fasting | — | Fed |
| Sequence 2 N = 6 | Fed | — | Fasting |

Twelve subjects will be randomized in 1:1 to receive one of the following on Day 1 of treatment:

After 10 hours of fasting, a single dose of 500 mg Compound 1C. No food is allowed for up to two hours after the dose (sequence 1, n=6).

After 10 hours fasting, a high-fat breakfast, to be ingested within 30 minutes. A single dose of 500 mg Compound 1C will be administered 30 minutes after the start of the meal (sequence 2, n=6).

After a 7-day washout period (Day 8), subjects in sequence 1 will be crossed-over to sequence 2, while subjects in sequence 2 will be crossed-over to sequence 1.

### Number of Patients:

Up to 18 healthy subjects (between 18 and 65 years of age, inclusive) will be included to have 12 evaluable subjects finishing both study sequence.

### Inclusion Criteria

The following criteria must be met by all subjects considered for study participation:

Subjects must be able and willing to give written informed consent.

Must be at least 18 years old at Screening and maximum 65 years old on date of first dose with a body mass index (BMI) of 19.0-34.0 kg/m2, inclusive at Screening.

If a woman of childbearing potential (WCBP), willing to undergo pregnancy testing and agrees to use two methods of birth control or is considered highly unlikely to conceive during the dosing period and for 3 months after last study treatment. WCBP must have a negative serum pregnancy test confirmed within 7 days prior to receiving the initial dose of combination therapy. (See Section P for addition details of acceptable contraception.)

If a male and sexually active with a WCBP, must agree to use effective contraception such as latex condom or is sterile (e.g., following a surgical procedure) and to refrain from sperm donation during the dosing period and for 3 months after last study treatment.

Must be medically healthy in the opinion of the Investigator as determined by pre-study medical history, vital signs, physical examination, laboratory tests and 12-lead ECG indicating no clinically significant abnormalities.

Must have the following clinical laboratory tests within the reference ranges or deemed not clinically significant by the Investigator: hemoglobin (Hgb), absolute neutrophil count, absolute lymphocyte count, platelet count, serum creatinine, alanine aminotransferase (ALT), aspartate aminotransferase (AST), alkaline phosphatase (Alk Phos) and bilirubin. Other laboratory tests that are abnormal should be discussed with the Medical Monitor.

Must be negative for hepatitis B surface antigen (HBsAg), hepatitis B core antibody, hepatitis C virus (HCV) antibody, and human immunodeficiency virus (HIV) I and II antibody tests at Screening.

Must be willing to refrain from alcohol and nicotine product consumption in the 48 hours prior to dosing and for the duration of the study.

Subjects must be negative for drugs of abuse and alcohol tests at Screening and admission on Day −1.

### Exclusion Criteria

Subjects will be excluded if they meet any of the following criteria:

The subject has a clinically relevant history or presence of respiratory, gastrointestinal, renal, hepatic, hematological, lymphatic, neurological, cardiovascular, psychiatric, musculoskeletal, immunological, dermatological, connective tissue diseases or disorders, or any condition known to interfere with absorption, distribution, metabolism or excretion of drugs.

Any acute illness or major surgery within the past 3 months determined by the Investigator to be clinically significant.

Clinically significant allergies which, in the opinion of the investigator, could interfere with participation in the study or known allergic reaction to any investigational product excipients.

History of hypertension or a blood pressure of >140 mmHg systolic or >90 mmHg diastolic at Screening.

A history of alcohol abuse or binge drinking or illicit drug use disorder within 3 years of Screening, as assessed by the Investigator.

Use of any tobacco- and/or nicotine-containing containing products, including cigarettes, e-cigarettes, vaping products, and nicotine replacement products, within 3 months of Screening.

Subjects will be excluded if they meet any of the following criteria:

The subject has a clinically relevant history or presence of respiratory, gastrointestinal, renal, hepatic, hematological, lymphatic, neurological, cardiovascular, psychiatric, musculoskeletal, immunological, dermatological, connective tissue diseases or disorders, or any condition known to interfere with absorption, distribution, metabolism or excretion of drugs.

Any acute illness or major surgery within the past 3 months determined by the Investigator to be clinically significant.

Clinically significant allergies which, in the opinion of the investigator, could interfere with participation in the study or known allergic reaction to any investigational product excipients.

History of hypertension or a blood pressure of >140 mmHg systolic or >90 mmHg diastolic at Screening.

A history of alcohol abuse or binge drinking or illicit drug use disorder within 3 years of Screening, as assessed by the Investigator.

Use of any tobacco- and/or nicotine-containing containing products, including cigarettes, e-cigarettes, vaping products, and nicotine replacement products, within 3 months of Screening.

Prolonged average QTcF (based on triplicate readings during Screening) of greater than 450 msec (for males) or 470 msec (for females).

The subject has clinically significant acute gastrointestinal symptoms at the time of Screening and/or admission (e.g., nausea, vomiting, diarrhea, heartburn).

Active or recent (<2 weeks ago) infection or known significant inflammatory process at Screening, including influenza, Covid. Subject should not be receiving active or prophylactic anti-infective treatment.

Use of prescription drugs within 30 days of first dosing unless agreed by the Investigator and Sponsor.

Use of aspirin or other non-steroidal anti-inflammatory agents (exception acetaminophen) or over the counter medication including herbal supplements within 7 days of first dosing, unless agreed as not clinically relevant by the Investigator and Sponsor.

The subject has donated and/or received any blood or blood products within the previous 90 days prior to first dosing.

Use of any inactivated vaccine within 14 days, or any live vaccine within 30 days prior to the start of study drug administration.

Any history of malignancy with the exception of completely resected (“cured”) localized skin squamous cell or basal cell carcinoma.

Subject cannot communicate reliably with the Investigator.

Subject is unlikely to comply with the requirements of the study.

Received an IRAK inhibitor within 3 months prior to the first dose of study drug.

Received an investigational medicinal product within 5 half-lives or within 30 days, whichever is longer, prior to the first dose of study drug or was exposed to more than 4 new chemical entities within 12 months prior to dosing of the study drug.

### Safety/Tolerability Parameters/Endpoints

The following are defined as safety/tolerability parameters:
Physical examination;
AEs;
Clinical laboratory assessments including hematology, serum chemistry, coagulation and urinalysis;
Vital signs;
12-lead ECG;

### Pharmacokinetic Parameters/Endpoints

PK parameters for Compound 1C, its active metabolite Compound 2, and the secondary metabolite Compound 3 will be estimated after each Compound 1C dose:
Maximum plasma concentration (Cₘₐₓ);
Time to Cₘₐₓ (tₘₐₓ);
Time from dose administration to the first measurable drug concentration (tlag);
Terminal elimination half-life (t_{1/2});
Area under the plasma concentration-time curve (AUC) from time of dosing (zero) to time t of the last measured concentration above the limit of quantification (AUC₀₋₄);
Area under the plasma concentration-time curve from time zero to infinity (AUC_{0-inf});
Percent extrapolated AUC (% AUCₑₓₜᵣₐₚ), i.e.,

$${AUC}_{0 - inf} - {AUC}_{0 - t} / {AUC}_{0 - inf} ;$$

Apparent total body clearance (CL/F);
Apparent volume of distribution (Vz/F);
Metabolic ratio (MR) of Compound 3/Compound 1C.

Additional parameters may be estimated, if deemed needed.

### Safety and Tolerability

Adverse events will be recorded and graded using CTCAE v.5 and a summary frequency table of adverse events will be provided. The severity and relationship to study drug [Compound 1C or placebo] of adverse events will also be summarized. Furthermore, if any serious adverse events occur, a brief summary of serious adverse events will be described and listed in tables.

Vital sign parameters will be listed with any values outside the normal range flagged. Descriptive statistics of absolute and change from baseline systolic and diastolic blood pressure, supine pulse rate, oxygen saturation and tympanic body temperature at each time point, will be tabulated by treatment.

Twelve-lead ECG parameters will be listed with any values outside the Wnormal range flagged. Descriptive statistics of absolute and change from baseline (pre-dose) heart rate, PR interval, QRS interval, QT interval, and QTcF interval at each time point, will be tabulated by treatment.

### Pharmacokinetic

Plasma concentrations of Compound 1C and its active and secondary metabolites Compound 2 and Compound 3 will be listed by dose level and summarized by time point and dosing day. The following descriptive statistics will be calculated: number of subjects (N), arithmetic mean, standard deviation (SD), median, minimum, maximum, coefficient of variation (CV %), geometric mean and CV % of geometric mean (GCV %). Nominal blood sampling times will be used in the summary.

PK variables will be summarized using number of subjects (N), arithmetic mean, standard deviation (SD), median, minimum, maximum, coefficient of variation (CV %), geometric mean and CV % of geometric mean (GCV %).

Exposure measurements (AUC0-t, AUC0-inf, and Cmax) will be log-transformed prior to any analysis. The 90 percent CI for the ratio of population geometric means between the fasting state (reference) and the fed state will be provided. An absence of food effect on bioavailability is established if the 90 percent CI for the ratio of population geometric means between fed and fasted treatments, based on log-transformed data, is contained in the equivalence limits of 80-125 percent for either AUC0-inf or Cmax. The clinical relevance of any difference in tmax and tlag will be evaluated.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for treating, managing, or ameliorating a myelodysplastic syndrome comprising administering to a subject having a myelodysplastic syndrome 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate, which has the following structure: 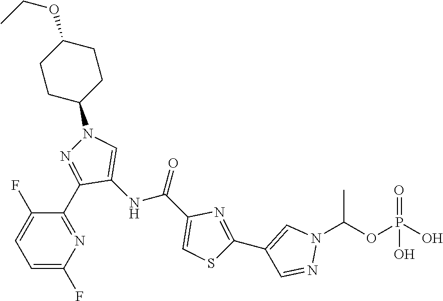 [SMILES: CCO[C@H]1CC[C@H](n2cc(NC(=O)c3csc(-c4cnn(C(C)OP(=O)(O)O)c4)n3)c(-c3nc(F)ccc3F)n2)CC1]
or a stereoisomer or mixture of stereoisomers, a pharmaceutically acceptable salt, tautomer, solvate, hydrate, co-crystal, clathrate, or polymorph thereof (Compound 1), wherein Compound 1 is orally administered to the subject in an amount of about 100 mg to about 2000 mg per day.

2. The method of claim 1, wherein Compound 1 is an inorganic base salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate, optionally Compound 1 is a mono-inorganic base salt or a di-inorganic base salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

3. The method of claim 1, wherein Compound 1 is an alkali metal salt, an alkaline earth metal salt, or an ammonium salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate, optionally Compound 1 is a mono-sodium salt, di-sodium salt, mono-potassium salt, di-potassium salt, calcium salt, magnesium salt, or mono-ammonium salt of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

4. The method of claim 1, wherein Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate disodium salt or 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate monosodium salt.

5. The method of claim 1, wherein Compound 1 is 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

6. The method of claim 1, wherein the myelodysplastic syndrome is at least one of the following:
(i) the myelodysplastic syndrome is a higher-risk myelodysplastic or a lower-risk myelodysplastic syndrome;
(ii) the myelodysplastic syndrome is a refractory or relapsed lower-risk myelodysplastic syndrome;
(iii) the myelodysplastic syndrome is a relapsed, refractory/resistant, intolerant, or has an inadequate response to one or more therapies for myelodysplastic syndrome;
(iv) the myelodysplastic syndrome is a lower-risk myelodysplastic syndrome that is relapsed, refractory/resistant, intolerant, or has an inadequate response to one or more therapies for lower-risk myelodysplastic syndrome;
(v) the myelodysplastic syndrome is a transfusion-dependent lower-risk myelodysplastic syndrome;
(vi) the myelodysplastic syndrome is a lower-risk myelodysplastic syndrome that is relapsed or refractory to erythropoiesis-stimulating agents;
(vii) the myelodysplastic syndrome is a lower-risk myelodysplastic syndrome that is relapsed or refractory to hypomethylating agents;
(viii) the myelodysplastic syndrome is a lower-risk myelodysplastic syndrome that is relapsed or refractory for treatment with erythropoiesis-stimulating agents and has previously been treated with one or more therapies for lower-risk myelodysplastic syndrome;
(ix) the myelodysplastic syndrome is a transfusion-dependent lower-risk myelodysplastic syndrome that is relapsed or refractory to erythropoiesis-stimulating agents;
(x) the myelodysplastic syndrome is a transfusion-dependent lower-risk myelodysplastic syndrome that is relapsed or refractory to hypomethylating agents;
(xi) the myelodysplastic syndrome is a transfusion-dependent lower-risk myelodysplastic syndrome that is relapsed or refractory to treatment with erythropoiesis-stimulating agents and has been previously received with one or more therapies for lower-risk myelodysplastic syndrome;
(xii) the myelodysplastic syndrome is ring sideroblast-negative; or
(xiii) the myelodysplastic syndrome is with del (5q) mutation and failed prior lenalidomide therapy.

7. The method of claim 6, wherein the one or more therapies comprise a treatment with an erythropoietin, luspatercept, or a hypomethylating agent, optionally wherein the hypomethylating agent is azacytidine or decitabine.

8. The method of claim 1, wherein Compound 1 is administered once daily or twice daily.

9. The method of claim 1, wherein the amount is about 100 mg to about 1000 mg per day.

10. The method of claim 1, wherein the amount is about 10 mg per day, about 125 mg per day, about 150 mg per day, about 200 mg per day, about 250 mg per day, about 300 mg per day, about 350 mg per day, about 375 mg per day, about 400 mg per day, about 450 mg per day, about 500 mg per day, about 550 mg per day, about 600 mg per day, about 650 mg per day, about 700 mg per day, about 750 mg per day, about 800 mg per day, about 850 mg per day, about 900 mg per day, about 950 mg per day, about 960 mg per day or about 1000 mg per day.

11. The method of claim 1, wherein the amount is about 125 mg twice daily, about 250 mg twice daily, about 400 mg twice daily or about 500 mg twice daily.

12. The method of claim 1, wherein the amount administered is:
(i) about 250 mg per day, administered in two doses, of about 125 mg each;
(ii) about 500 mg per day, administered in two doses, of about 250 mg each;
(iii) about 800 mg per day, administered in two doses, of about 400 mg each;
(iv) about 1000 mg per day, administered in two doses, of about 500 mg each;
(v) about 750 mg per day administered in two doses, wherein a first dose is about 500 mg and a second dose is about 250 mg; or
(vi) about 750 mg per day administered in two doses, wherein a first dose is about 250 mg and a second dose is about 500 mg.

13. The method of claim 11, wherein the two doses are administered at an interval of about 6-15 hours.

14. The method of claim 1, wherein Compound 1 is administered in an amount corresponding to about 250 mg per day, about 500 mg per day, about 750 mg per day, about 125 mg twice per day, about 250 mg twice per day, about 400 mg twice per day or about 500 mg twice per day of 1-(4-(4-((3-(3,6-difluoropyridin-2-yl)-1-((1r,4r)-4-ethoxycyclohexyl)-1H-pyrazol-4-yl)carbamoyl)thiazol-2-yl)-1H-pyrazol-1-yl)ethyl phosphate.

15. The method of claim 1, wherein Compound 1 is administered as an oral tablet.

16. The method of claim 15, wherein the oral tablet comprises:
a) about 15% to about 35% Compound 1 by weight based on total weight of the tablet;
b) about 50% to about 70% intra-granular excipient by weight based on total weight of the tablet; and
c) about 10% to about 20% extra-granular excipient by weight based on total weight of the tablet;
optionally the oral tablet comprises about 10 to 300 mg of Compound 1.

17. The method of claim 1, further comprising administering a therapeutically effective amount of a second active agent or a supportive care therapy.

18. The method of claim 1, wherein the subject is 18 years or older, optionally wherein
the subject has failed prior lenalidomide therapy, or
the subject has del (5q) mutation and has failed prior lenalidomide therapy.

19. The method of claim 1, wherein Compound 1 is administered with food.

20. The method of claim 1, wherein Compound 1 is administered without food.
